(19) United States
(12) Patent Application Publication
(10) Pub. No.: US 2024/0082053 A1
(43) Pub. Date: Mar. 14, 2024

(54) MONOLAYER CELL PATCH IN AN EXTRACELLULAR MATRIX SCAFFOLD

(71) Applicant: Carnegie Mellon University, (US)

(72) Inventors: Adam Walter Feinberg, Pittsburgh, PA (US); Rachelle Simko, Pittsburgh, PA (US)

(21) Appl. No.: 17/769,626

(22) PCT Filed: Oct. 19, 2020
(86) PCT No.: PCT/US2020/056327

§ 371 Date: Apr. 15, 2022

(51) Int. Cl. A61F 9/00; A61K 35/30; A61L 27/18; A61L 27/34; A61L 27/36; A61L 27/38; A61L 27/50; C12N 5/079

(52) U.S. Cl. CPC A61F 9/0017; A61K 35/30; A61L 27/18; A61L 27/34; A61L 27/3633; A61L 27/3808; A61L 27/507; C12N 5/0621; A61F 2250/0067; A61L 2300/414; A61L 2300/62; A61L 2300/64; A61L 2430/16; C12N 2533/90

(57) ABSTRACT

A process for micro-tissue encapsulation of cells includes coating a tissue scaffold stamp with an extracellular matrix compound. The process includes depositing the tissue scaffold stamp onto a thermoresponsive substrate and seeding the tissue scaffold stamp with a cell culture. A cell culture forms a cell patch that is attached to the extracellular matrix compound. A monolayer on the tissue scaffold stamp for which borders of the monolayer maintain expressions for cell-cell junctions, wherein the cell-cell junctions of the monolayer are configured to express tension forces. The process includes removing the thermoresponsive substrate. The process includes folding the micro-tissue structure by suspending the micro-tissue in the solvent. The folded micro-tissue structure is collected from the solvent and administered to an organism.

### CLAIM OF PRIORITY

This application claims priority under 35 U.S.C. § 119(e) to U.S. Patent Application Ser. No. 62/973,695, filed on Oct. 18, 2019, the entire contents of which are hereby incorporated by reference.

### GOVERNMENT RIGHTS

This invention was made with government support under Grant No. 1R01EY024642-01A1, awarded by the National Institutes of Health. The government has certain rights in the invention.

### FIELD OF THE TECHNOLOGY

This disclosure relates to the formation of micro-tissues and of application of the micro-tissues to organisms.

### BACKGROUND

Administration of cells to patients has become a promising therapy for many diseases. However, cells delivered in single cell suspension often die quickly after being injected into the site for repair and it is also unknown if single cells can adequately repair damaged or failing organs and tissues that are composed of millions of cells. Current methods for cell injection therapy involve using enzymatic release of cells into a single cell suspension which causes changes in their cellular structure and phenotype. Additionally, injected single cells often die after injection from a combination of stress induced from the physical injection process and lack of attachment to the desired tissue.

### SUMMARY

The systems and processes described in this document allow for the formation of small intact cell patches (e.g., micro-tissues) and application of the micro-tissues to a body of a patient. The thermal release of these micro-tissues from a substrate (e.g., an extracellular matrix protein scaffold (ECM)) on which the cell patches are cultured allows for the cells to maintain their structure and phenotype, which allows the cells to repair damaged tissue after delivery. The extracellular matrix is configured to surround the cultured cell patch (e.g. a monolayer of cells) to protect the cell patch from physical stress of a delivery process to the body. The ECM also provides an attachment site that facilitates attachment of the cell patch to a desired target tissue (e.g., for tissue repair).

The ECM can include encapsulation materials that replicate a micro-environment desired for the cell patch in vivo. For example, the ECM is configured to be similar in density, structure, and/or composition to the native ECM these cells are surrounded by in vivo. The ECM provides a unique microenvironment that more closely matches that found in vivo and thus improves the ability to modulate cell behavior.

The cell patch can include a cell monolayer. The monolayer is formed to include cell expressions for cell-cell tight junctions in the cell patch. A relatively larger substrate and longer culture time (e.g., −24 hours) enable tight junctions to form. These junctions generally appear in vivo and enable the cell patch to be more readily accepted into tissue when administered.

The shrink-wrapped monolayers provide one or more of the following advantages. Biological features of the cells are maintained for in vivo administration, in contrast to enzyme-based approaches to releasing cells from a scaffold. The cells maintain their phenotype (e.g., tight junctions) in the monolayer. The cells form patches of empithelium and/or build a cytoskeleton that can be maintained for cell administration. For example, when introduced in vivo in a variety of contexts (e.g., in the heart, cornea, or for lung repair), cells integrate into tissue easily, as subsequently described. Specifically, the cell-cell junctions are maintained.

The cell patches can be used for treating diseases, as subsequently described. For example, cornea repair can be performed using cell monolayers. Cardiac repair can be performed. In some implementations, lung repair can be performed.

As subsequently described, monolayers provide other advantages with respect to single cell approaches. For example, the endothelium and/or epithelium are barriers for cell administration. Because the cells monolayers being administered are form cell patches, the endothelium allows the patches to integrate. In some implementations, the cell patch attaches as a dome of cells, and flattens and integrates over time with the endothelium. Additionally, monolayer cells migrate out from patch to rest of endothelium, which does not occur when individual cells are applied. Individual cells are less likely to attach. The monolayers thus result in increased survival with respect to single cells. The cell junctions can affect cell signaling to promote cell adhesion in this way, which increases viability, as the cell phenotype is maintained.

In some implementations, the cells additional materials are added to the extracellular matrix to facilitate patterning. For example, antibodies can be added to deliver to a specific location (e.g., a targeted delivery). In some implementations, growth factors can be added to the matrix that is shrink wrapped (e.g., pbGF for muscle cells). This can enhance, for example, vascular ingrowth which is needed for muscle cells. In some implementations, this approach includes labeling for validation. For example, cell tracker (e.g., cytoplasmic dyes) can be used.

In some implementations, different shapes of scaffolds are possible. In addition to squares, tubes can be generated to generate tubules. This can facilitate growth of fragments of blood vessels and/or chains of endothelial cells. In some implementations, it is possible to mix different kinds of shrinkwrapped cells.

Various disease treatments are possible using the shrinkwrapped cells. For example, in some embodiments, described herein includes injecting cells into patients that suffer from corneal blindness due to low cell density. In some embodiments, described herein includes the injection of micro-monolayers that can increase the cell density without the need to remove the existing endothelium.

In some embodiments, described herein includes the integration of corneal endothelial cell micro-monolayers that can be injected through a small gauge need to help alleviate density driven corneal blindness.

In some embodiments, described herein includes an array of cell assemblies that can be created by an extracellular matrix shrink wrapped cells (SHELL) technique, wherein upon thermal release, the cell assemblies can maintain their cell-cell junctions and can contract into a size small enough to be injected into tissues through a needle.

In some embodiments, described herein includes an in vivo injection process that involves maintaining an injected eye down after injection to allow for integration of the cell assembly into the corneal endothelium. In some embodiments, described herein includes the synthesis of an extracellular matrix SHELL scaffold that can help maintain the cells in a monolayer and can maintain the cytoskeleton of the cell in the monolayer, which together with their cell-cell junctions can improve integration.

In an aspect, a process for micro-tissue encapsulation of cells includes coating a tissue scaffold stamp with an extracellular matrix compound. The process includes depositing the tissue scaffold stamp onto a thermoresponsive substrate. The process includes seeding the tissue scaffold stamp with a cell culture. The process includes incubating the cell culture on the tissue scaffold stamp at a temperature that is specified, wherein the cell culture forms a cell patch that is attached to the extracellular matrix compound. The process includes forming, by the cell patch, a monolayer on the tissue scaffold stamp in which borders of the monolayer maintain expressions for cell-cell junctions. The process includes removing the thermoresponsive substrate. The process u) includes removing the tissue scaffold stamp from the cell patch to form a micro-tissue structure around the cell patch. The process includes folding the micro-tissue structure by suspending the micro-tissue in the solvent. The process includes collecting the folded micro-tissue structure from the solvent. The process includes administering the folded micro-tissue structure to an organism.

In some implementations, the process includes forming the tissue scaffold into a tube configuration. In some implementations, the process includes forming a cell patch comprising a tube geometry based on the tube configuration of the tissue scaffold.

In some implementations, the cell patch comprises a fragment of a blood vessel. In some implementations, the process includes adding antibodies to the cell patch. In some implementations, administering the micro-tissue structure to an organism comprises injecting the micro-tissue structure. In some implementations, the cell patch comprises corneal endothelial cells. The process includes introducing the cell patch to a cornea and ensuring a contact of the cell patch with the cornea using gravity.

In some implementations, a size of the micro-tissue structure is proportional to a size of the tissue scaffold stamp, and wherein the size of the micro-tissue structure is a fraction of a diameter of an injecting apparatus. In some implementations, the tissue scaffold stamp comprises an organosilicon compound. In some implementations, the organosilicon compound comprises Polydimethylsiloxane. In some implementations, the extracellular matrix compound comprises a protein comprising one or more of collagen IV, laminin, a fibroblast growth factor protein, and a vascular endothelial growth factor protein.

In some implementations, depositing the tissue scaffold stamp comprises printing the tissue scaffold stamp onto the thermoresponsive substrate. In some implementations, the thermoresponsive substrate comprises a PIPAAm polymer. In some implementations, the tissue scaffold stamp forms a regular geometry. In some implementations, the tissue scaffold stamp comprises a surface dimension of less than or approximately equal to 250 μm². In some implementations, the cell patch comprises between 10 and 100 cells.

In a general aspect, a system includes a cell patch comprising a cell monolayer that maintains expressions for cell-cell junctions and cytoskeletons for cells that are in the cell patch. The system includes a micro-tissue structure folded around the cell patch, the micro-tissue structure comprising an extracellular matrix configured to provide a physical barrier between the cell patch and an external environment.

In some implementations, the extracellular matrix comprises a protein comprising one or more of collagen IV, laminin, a fibroblast growth factor protein, and a vascular endothelial growth factor protein. In some implementations, the micro-tissue structure forms a tube configuration. In some implementations, the monolayer comprises between and 100 cells. In some implementations, the cell patch comprises muscle tissue. In some implementations, a growth factor is added to the extracellular matrix to promote vascular ingrowth of the muscle tissue.

The details of one or more embodiments of these systems and methods are set forth in the accompanying drawings and the description to be presented. Other features, objects, and advantages of these systems and methods will be apparent from the description and drawings, and from the claims.

### DESCRIPTION OF DRAWINGS

FIGS. 1A-1B show processes for extracellular matrix encapsulation.

FIGS. 2-8 show images of extracellular matrix encapsulation using tubule scaffolds.

FIGS. 9-14 show images and graphs for extracellular matrix encapsulation for monolayer corneal endothelial cell cultures.

FIG. 15 shows a process for extracellular matrix encapsulation and administration.

### DETAILED DESCRIPTION

The extracellular matrix (ECM) described herein includes an array of geometrical shapes that will fold upon release. For example, the ECM can form one or more patterned geometries that have micrometer dimensions for length and width and nanometer thickness. In some examples, the geometrical shapes in the array include extracellular matrix proteins that can be used to culture cells that allow for the formation of 2D micro-tissues, specifically cell monolayers. These micro-tissues can then be released from a substrate upon which they are formed. In some implementations, the micro-tissues are thermally released. Upon release, these micro-tissues spontaneously fold up such that the ECM forms an outer layer around the cultured cells, and the cultured cells retain their micro-tissue structure and phenotype. The folded micro-tissues can then be administered, for example by injection through a needle, to repair or replace tissues. The micro-tissues have in vivo and in vitro applications. For example, the micro-tissues support the formation of corneal endothelial cell micro-tissues that can be injected through a small gauge needle.

The processes and systems described herein include encapsulation of micro-monolayer cell patches (e.g., μMonolayers) in a thin layer of ECM protein that allows cells to maintain high viability, cell-cell junctions, and cytoskeletal structure post-injection, while also providing the cells with cell-ECM interact ions that can promote cell adhesion. In vitro results confirm that the CE cells formed a monolayer on the engineered ECM substrates and within 24 hours had established tight junctions and formed an organized F-actin cytoskeletal structure, which was retained through the thermal release and injection process. When compared to enzymatically-released single cells, injected shrink-wrapped μMonolayers significantly increase CE monolayer cell density, as subsequently described.

Additionally, shrink-wrapped μMonolayers attached to both in vitro mono layers and ex vivo corneas within 3 hours, indicating that the described encapsulated processes could be applied using the protocols that are currently in use in clinical trials for single cell CE inject ion. For example, in vivo rabbit studies utilizing healthy rabbit eyes showed high numbers of shrink-wrapped μMonolayers integrated into the healthy CE. Generally, cells within a young healthy rabbit CE are contact-inhibited and are at an extremely high density. Therefore, if the shrink-wrapped μMonolayers are able to integrate within such a tissue, integration into damaged or diseased CEs with a much lower cell density occur at much higher rates.

Generally, the ECM includes a fibrillar network of proteins, glycosaminoglycans and other biomolecules. The ECM forms a scaffold around cells that provides, for example, structural support, growth factor sequestration, a network for adhesion and mechanical signaling, and a host of other functions. The ECM can function as an environment, or niche, that is suitable for the functioning of the cultured cells of the micro-tissues. For example, the adult stem cell niche includes a unique ECM protein structure, composition, support cell population and set of soluble and insoluble signaling molecules that help maintain the multipotent state of the stem cells. The ECM is an artificially produced protein matrix, rather than a naturally-occurring ECM. The selection of ECM proteins is chosen based on the cell culture being produced, as described in further detail below.

In some examples, 2D culture cells are typically grown on rigid tissue culture treated polystyrene (TCPS) that is pre-coated with an ECM protein or coated with ECM proteins that are included in a serum supplemented into the media. While such ECM proteins enable adhesion of cells to the TCPS and subsequent proliferation, many primary cell types can only be passaged a limited number of times before becoming senescent of changing phenotype, such as undergoing epithelial to mesenchymal transition (EMT). Culture in 3D using synthetic and/or natural hydrogels can address some of these limitations by altering the chemo-mechanical environment to better replicate in vivo conditions and have been effective for culturing a wide range of cell types. However, these hydrogels are typically isotropic in structure, do not recreate ECM dense structures such as basement membranes and have compositions (e.g., collagen, fibrin, matrigel, PEG) that typically differ from that of the complex in vivo environment. Further, passaging these cells, whether in 2D or 3D, often requires using enzymes and calcium chelators that disrupt cell-matrix and cell-cell adhesion to produce a single cell suspension. When re-seeded the cells must expend energy to reestablish cell matrix and cell-cell adhesions in the new environment into which they are placed. The ECM is configured to mimic a cell micro-environment that is found in vivo by (i) encapsulating cells in a defined ECM that better mimics the native ECM structure and (ii) minimally disrupting cell-matrix and cell-cell adhesions.

ECM nano-scaffolds are formed that can be used to at least partially encapsulate cells in order to modulate the chemo-mechanical microenvironment. Using an adaptation of surface-initiated assembly (SIA), well-defined nano-scaffolds of assembled ECM proteins are formed into free standing structures. By adhering cells prior to the release of these ECM nano-scaffolds, the cells are encapsulated (e.g., shrink-wrapped), in a layer of assembled protein matrix. In some implementations the ECM nano-scaffolds are engineered at the size scale of the cell, ˜75 μm in lateral dimensions and ˜50 nm thick. In some implementations, the SIA approach can be used to encapsulate a variety of cell types in defined ECM including one or more of fibronectin (FN), laminin (LAM), fibrinogen (FIB) and collagen type IV (Col IV), representing the major protein composition of the native pericellular matrix. The long-term goal is that these ECM nano-scaffolds and the encapsulation process will enhance therapeutic cell delivery by supporting survival and functional integration of cells in an otherwise diseased matrix environment, such as that found in infarcted myocardium.

The ECM nano-scaffolds can be used with any adherent cell type and could even be extended to non-adherent cells if antibodies for cell surface markers are mixed in the ECM protein solution before incubating on the PDMS stamp. For example, cell types can include hepatocytes, which includes an adherent cell type. For example, cell types can include killer t cell, which are a non-adherent cell-type, and are combined with a cell surface marker antibody in the ECM. These cell types can be used with collagen I, collagen IV, fibronectin, laminin, vitronectin, and any ECM protein that can be micro-contact printed.

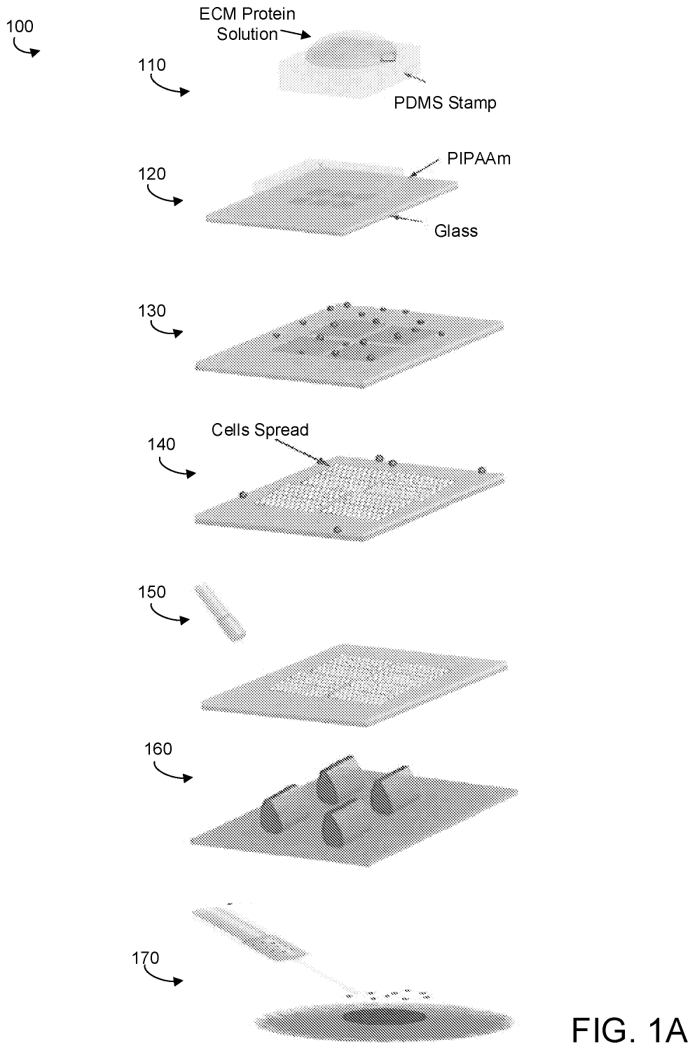

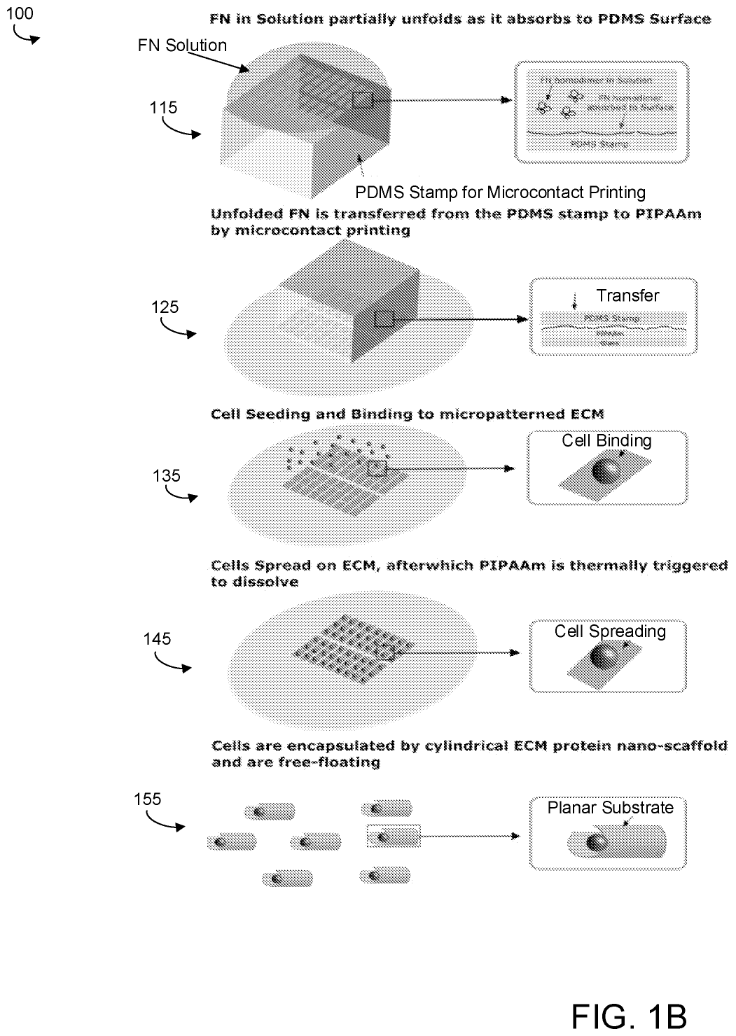

FIG. 1A shows an example process 100 for shrink-wrapping and injecting corneal endothelial encapsulated cell patches (e.g., cell monolayers). Surface-initiated assembly techniques are used to engineer 200 micrometer (μm) by 200 μm by 5 nanometer (nm) ECM scaffolds on the thermoresponsive polymer, PIPAAm. The samples and cells are then heated to 40° C. before seeding the cells on the squares and culturing for approximately 24 hours. After approximately 24 hours, samples are rinsed with warm media and cooled to room temperature to trigger the dissolution of the PIPAAm and shrink-wrapping/release of the micron sized monolayers of the cell patches (e.g., corneal endothelial cells).

The process 100 can be performed for each of the following embodiments.

ECM scaffold fabrication The ECM scaffolds were fabricated via previously described surface-initiated assembly with minor modifications (e.g., shrink wrap paper). Briefly, 1 centimeter (cm) by 1 cm PDMS stamps designed to have 200 μm by 200 μm square features were fabricated via standard soft lithography techniques. The stamps were sonicated in 50% ethanol for 60 minutes, dried under a stream of nitrogen and incubated for 60 minutes with a 50:50 mixture of 50 μg/mL collagen IV (COL4) and 50 μg/mL laminin (LAM), as shown in step 110. Either 50% AlexaFluor 488 labeled COL4 or 50% AlexaFluor 633 labeled LAM was used to visualize the protein. Following incubation the stamps were rinsed in sterile water, dried under a stream of nitrogen and brought into conformal contact with poly(N-isopropylacrylamide) (PIPAAm) (2% high molecular weight, Scientific Polymers) coated 25 mm glass coverslips for 30 minutes to ensure transfer of the squares, as shown in step 120. ECM square micro-contact printed on PDMS coverslips were used as controls. Upon stamp removal, laser scanning confocal microscopy was used to determine the quality of the transferred ECM squares (Nikon AZ I 00).

### Example Embodiment: Bovine Corneal Endothelial Cell Culture

Bovine corneal endothelial cells (BCECs) were isolated and cultured as previously described (ref EBM and expansion paper). Briefly, corneas were excised from the whole globe (Pel Freez), incubated endothelial side up in a ceramic 12 well spot plate with 400 μL of TrypLE Express for 20 minutes. The cells were then gently scraped from the cornea using a rubber spatula, centrifuged at 1500 RPM for 5 minutes, re-suspended in 5 mL of culture media (low glucose DMEM with 10% FBS, 1% Pen/Strep/AmphB and 0.5% gentamicin, designated at P0 and cultured in a 50 kPa PDMS coated T-25 flask that was pre coated with COL4. Fifty whole eyes were received at a time and were used to seed 5 T-25 flasks. Cells were cultured until confluence and split 1:3 until they were used once confluent at P2.

Shrink-Wrapping BCEC uMonolayers in ECM Scaffolds

Patterned coverslips (25 mm) were secured with vacuum grease in the bottom of 35 mm petri dishes which were placed on dry block set to 52° C. This resulted in the coverslips reaching (within 30 min) and holding at 40° C. Bovine CECs were released from the culture flask with TrypLE Express, centrifuged and re-suspended at a density of 150,000 cells/mL in 15 mL centrifuge tubes. The tubes were placed in a dry block set at 45° C. for approximately 5 minutes, or until the cell solution just reached 40° C. and 2 mL of cell suspension was added to each 35 mm dish before it was immediately placed in an incubator (37° C., 5% CO₂). Cells were cultured on the squares for 24 hours to allow them to form small monolayers on the 200 μm squares. After 24 hours, samples were removed from the incubator, rinsed twice in 37° C. media, 2 mL of fresh warm media was added, and the sample was allowed to cool to room temperature. Once the temperature decreased <32° C. the PIPAAm dissolved and released the scaffolds+μMonolayers. The release process was recorded using a Photometrics CoolSnap camera. Following release the scaffolds+μMonolayers were collected via centrifugation at 1500 rpm for 5 minutes before use in further experiments. CECs seeded on to PDMS coverslips were used as a control.

Immunostaining of shrink-wrapped BCECs: Shrink-wrapped μMonolayers re-suspended in PBS containing Ca2 and Mg2 (PBS++) were injected through a small gauge needle onto a glass coverslip and allowed to settle for about 15 minutes before fixation for 15 minutes in 4% paraformaldehyde (PBS++). Samples were gently washed 2×'s with PBS++ and incubated with 1:100 dilution of DAPI, 1: I 00 dilution of mouse anti-ZO-1 antibody (Life Technologies) and 3:200 dilution of AlexaFluor 488. Samples were rinsed 2 times for 5 minutes with PBS++ and incubated with 1:100 dilution of AlexaFluor 555 goat anti-mouse secondary antibody for about 2 hours. Samples were rinsed 2 times for 5 minutes with PBS++, mounted on glass slides with Pro-Long Gold Antifade (Life Technologies) and then imaged on a Zeiss LSM 700 confocal microscope.

Viability of Shrink-Wrapped BCECs Post-Injection

After centrifugation, shrink-wrapped μMonolayers or TrypLE Express released single cells were re-suspended in 200 μL of growth media, drawn up into a 280 needle, injected into a petri dish and incubated with 2 μM calcein AM and 4 μM EthD-1 (Live/Dead Viability/Cytoxicity Kit, Life Technologies) in PBS++ for 30 minutes at 37° C. After 30 minutes, samples were imaged on a Zeiss LSM 700 confocal; 5 images per sample and 3 samples per type were used. The number of live and dead cells was counted manually. The number of live cells was divided by the number of dead cells to determine the percent viability of both the ECM scaffold wrapped cells and enzymatically released cells.

Seeding of Shrink-Wrapped and Single BCECs on Stromal Mimics

Self-compressed collagen I films were prepared as previously described. Briefly, a 6 mg/mL collagen I gel solution was prepared per manufacturer's instructions and pipetted into 9 mm diameter silicone ring molds on top of glass coverslips. The gels were placed into a humid incubator (37° C., 5% CO2) for 3 hours to compress under their own weight. The gels were then dried completely in a biohood followed by rehydration in PBS, forming a thin collagen I stromal mimic. Shrink-w rapped BCEC μMonolayers were seeded onto the films at a 1:1 ratio of stamped coverslip to collagen I film. As a control, BCECs that were cultured in the flasks and enzymatically released using TrypLE Express into a single cell suspension were seeded onto collagen I films. The number of control cells seeded was equal to the number of cells that would be seeded from one stamped ECM scaffold sample if all squares had full monolayers on them, as a best case scenario. The average number of cells that occupied the 200 μm square was 30 cells; so cells×1600 squares per stamp, meaning approximately 48,000 cells per sample. Therefore, 50,000 cells per sample were seeded for the controls. At 6 and 24 hours, samples were removed from culture and fixed and stained for the nucleus, ZO-1 (tight junction protein) and F-actin.

Briefly, samples were rinsed twice in PBS++, fixed in 4% parafolmaldehyde (in PBS++) with 0.05% Triton-X 100 for 15 minutes. Samples were rinsed twice for 5 minutes with PBS++ and incubated with 5 drops of NucBlue (Life Technologies) for 10 minutes. Samples were rinsed once with PBS++ and incubated with 1:100 dilution of mouse anti-ZO-1 antibody (Life Technologies) and 3:200 dilution of AlexaFluor 488 or 633 phalloidin for 2 hours. Samples were rinsed 3 times for 5 minutes with PBS++ and incubated with 1:100 dilution of AlexaFluor 555 goat anti-mouse secondary antibody for 2 hours. Samples were rinsed 3 times for 5 minutes with PBS++, mounted on glass slides using Pro-Long Gold Anti-fade and imaged on a Zeiss LSM 700 confocal microscope.

In Vitro Integration of Shrink-Wrapped Vs Single Bovine CECs

To mimic a low-density aging CE, 25,000 PS bovine cells were seeding onto the collagen I stromal mimics as described above, until confluent to form the “aged” monolayers. Shrink-wrapped μMonolayers and single BCECs were prepared as previously described, labeled with CellTracker Green (Life Technologies) for 30 minutes, centrifuged, diluted to the equivalent of 50,000 cells/sample and injected on to the “aged” monolayers. “Aged” monolayers with no cells injected on top served as controls. Samples were fixed at stained at days 3, 7 and 14 as described above. A Zeiss LSM700 confocal was used to image 10 random spots on each sample and the cell density was manually counted (e.g., cell nuclei). The number of nuclei was divided by the image area to obtain the cells/mm 2 per image. The cell density for each sample was determined by averaging the cell densities of each image and the average cell density of each sample type was determined by averaging the cell density of the 3 or 4 samples. To examine the outgrowth of the shrink-wrapped μMonolayers over time, confocal images centered around and individual shrink-wrapped μMonolayer (day 3 n=33, day 7 n=37, day 14 n=40) were collected and the CellTracker channel was converted in to a binary black and white image. The binary images for each sample type were then converted in to a Z-stack and analyzed via the Heat Map for Z-stacks plugin (relative without log 10) to determine the average pixel density of CellTracker.

Live Imaging of In Vitro Integration of Shrink-Wrapped Bovine CECs

For live imaging, the “aged” monolayer on the collagen I stromal mimic, was first incubated for 30 minutes with CellTracker Orange to differentiate between the existing monolayer and injected cells, which were labeled with CellTracker Green as described above. HEPES buffered Opti-MEM I Reduced Serum Media (Life Technologies) with 10% FBS, 1% Pen/Strep was added to the monolayer and Shrink-wrapped μMonolayers that were prepared as described above were injected through a 30G needle on top of the sample. The sample was placed on the Zeiss LSM700 confocal equipped with a temperature chamber set to 37° C. for 30 minutes to allow for the cells to settle. Using the Definite Focus system, a time lapse series of one z-stack was obtained every hour for 48 hours.

Rabbit CEC Isolation, Culture and Shrink-Wrapping

Whole rabbit eyes were received on ice from Pel Freez Biologicals. Corneas were excised from the whole globe, the CE and Descemet's Membrane were manually stripped with forceps and incubated in Dispase (1U/mL, Stem Cell Technologies) for 1.5 hours at 37° C. to detach the rabbit CE cells (RCECs) from the Descemet's Membrane. The RCECs were then gently pipetted up and down, diluted in culture media (DMEM/F12, 10% FBS, 0.5% Pen/strep), centrifuged at 1500 RPM for 5 minutes, re-suspended in 10 mL of culture media, designated at P0 and cultured COL4 coated T-25 flasks with the equivalent of 15-25 eyes per flask depending on cell yield. RCECs were cultured until confluence and split 1:2 and used in all experiments once confluent at P1 or P2. RCEC μMonolayers were shrink-wrapped as described above with the following modifications: ECM scaffolds (using the same 1 cm×1 cm PDMS stamps) were stamped on to 18 mm glass coverslips to avoid having excess seeding area and reduce the number of cells that need to be seeded per sample to still achieve full coverage with −50,000 cells/sample once confluent. Coverslips were secured via vacuum grease to the bottom of Nunc IVF center well dishes (20 mm diameter inner well), cells were re-suspended at 150,000 cells/mL and 1 mL of RCECs was seeded per sample.

Ex Vivo Integration of Shrink-Wrapped Rabbit CECs

Three whole rabbit eyes were placed cornea up in a 12-well plate. Shrink-wrapped RCEC μMonolayers were prepared as described above. Two samples of μMonolayers per ex vivo eye were prepared and re-suspended in 100 μL of DMEM/F12. A 30-G insulin syringe was used to draw up the full I 00 μL, the needle was inserted in to the center of the cornea until it was visible in the anterior chamber and 504, of the suspension was injected. This resulted in the equivalent of 50,000 cells injected into the anterior chamber. The needle was held in place for a few seconds to ensure the media and cells did not come back out of the injection site. The injection was viewed under a stereomicroscope and the pink color of the media filling the anterior chamber was visible, indicating successful injection. The eyes were flipped and incubated cornea down for 3 hours at 37° C., 5% CO2 in a humidified incubator. After 3 hours, the whole eye was placed in 2% paraformaldehyde (PBS++) at 4° C. for 24 hours. After 24 hours the eye was rinsed in PBS and the cornea was excised and rinsed 3×'s for 5 minutes. The cornea was then incubated CE facing down on 1 mL of PBS++ containing 2 drops of NucBlue (Life Technologies), 2:100 dilution of mouse anti-ZO-1 antibody (Life Technologies) and 3:200 dilution of AlexaFluor 488 Phalloidin (Life Technologies) for 2 hours at room temperature. Corneas were then rinsed 3×'s for 5 minutes in PBS followed by 2 hour incubation on 1 mL PBS++ with 2:100 dilution of AlexaFluor 555 goat anti-mouse secondary antibody for 2 hours and stored in PBS before imaging on the Zeiss LSM700 confocal.

In Vivo Injection and Integration of Shrink-Wrapped CECs

Shrink-wrapped RCEC μMonolayers were prepared as described above with one minor modification: cells were labeled with Vybrant DiO 1 day prior to seeding on to the ECM nano-scaffold s by incubating cells in 1 mL of media with 5 μL of Vybrant DiO for 30 minutes followed by 3 ten minute rinses with fresh media. An excess number of μMonolayer samples were prepared (16) in order to ensure there was enough volume for injection. The Shrink-wrapped μMonolayers were released and were re-suspended in one tube 1.5 mL micro-centrifuge tube in a total of 300 μL. Three female New Zealand white rabbits with healthy intact CEs weighing approximately 2.5 kg were used for this study. Rabbits were anesthetized with Ketamine (40 mg/kg) and Xylazine (4 mg/kg) followed by isofluorene to keep rabbits under sedation for 3 hours. Rabbit #1 was injected in right eye (OD) with 504, (˜100,000 cells). Rabbit #2 was initially injected with 1004, in to the right eye, however most of the volume came back out of the cornea, so an additional 504, was injected in to a second location for 300,000 cell injection if all cells had successfully stayed within the anterior chamber. Rabbit #3 had a smaller than usual right eye so the left eye was injected with 1004, or approximately 200,000 cells. Immediately after injection, each rabbit was placed on their side with the injected eye facing down for 3 hours to ensure attachment of the cells. On day 7, rabbits were anesthesized with an intramuscular injection of ketamine (40 mg/kg) and xylazine (4 mg/kg) and then euthanized with of Euthasol solution (1 mL/1.8 lbs.) containing (390 mg/mL Sodium Pentobarbitol, 50 mg/mL Phenytoin Sodium) through an ear vein injection. Eyes were immediately enucleated and intravitreal injected with 100 μL 2% paraformaldehyde (PBS++).

The whole eye was then immersed in 2% paraformaldehyde (PBS++) and fixed at 4° C. for 24 hours. After 24 hours the eye was rinsed in PBS and the cornea was excised and rinsed 3×'s for 5 minutes. The cornea was then incubated CE facing down on 1 mL of PBS++ containing 2 drops of NucBlue (Life Technologies) and 2:100 dilution of mouse anti-ZO-1 antibody (Life Technologies) for 2 hours at room temperature. Corneas were then rinsed 3×'s for 5 minutes in PBS followed by 2 hour incubation on 1 mL PBS++ with 2:100 dilution of AlexaFluor 555 goat anti-mouse secondary antibody for 2 hours and stored in PBS before imaging on the Zeiss LSM700 confocal.

### Example Embodiment: Tubule ECM Structures for Cardiac Tissues

In an aspect, the tubule endothelial segments are generated by culturing endothelial cells on micropatterned fibronectin rectangles on PIPAAm, a thermosensitive polymer. Following dissolution of the PIPAAm, tubule segments are released, which are able to be incorporated into engineered cardiac tissues to create a primitive vascular network. Fibronectin rectangles are stamped onto PIPAAm. Endothelial cells are seeded on three different stamp patterns form a confluent monolayer on the rectangles at different seeding densities. Endothelial tubules are released from the PIPAAm surface using thermal control.

The tubule endothelial segments can be configured for treatments of cardiovascular disease (CVD). CVD is a significant global concern and the leading cause of mortality worldwide. CVD includes myocardial infarction, heart failure, and congenital heart defects, and can lead to the inability of the heart to supply blood to tissues throughout the body. Recent advances in tissue engineering have produced three-dimensional cardiac tissues from fibroblasts in combination with embryonic or induced pluripotential stem cell-derived cardiomyocytes. These engineered cardiac tissues could potentially be used to “patch” damaged areas of the heart. However, the heart is an extremely complex organ with multiple cellular types present, in addition to both fibroblasts and cardiomyocytes. The cell patches described here are configured to provide a supply of vascular tissues.

FIG. 1B shows a process 105 for forming the endothelial tubule segments. The endothelial tubule segments are generated in three-dimensions and can be incorporated into engineered cardiac constructs. This would allow for the increase in size of viable tissue engineered transplants which may have a higher capability of fixing damaged hearts. The dimensions of micro-patterned rectangles and concentrations of HUVECs to create the most effective 3D endothelial tubule segments were determined as described below. Fibronectin rectangles were micropatterned onto a thermosensitive polymer, Poly(N-isopropylacrylamide) (PIPAAm). Endothelial cells were then cultured on these fibronectin rectangles and allowed to form tight junctions, which are important cell-cell junctional components found in tissue vasculature. Tubule segments consisting of the fibronectin and endothelial cells were then released following PIPAAm dissolution.

As shown in FIG. 1B, at step 115, PDMS stamps are coated in labeled fibronectin and then dried. At step 125, PDMS stamps are placed stamp-side-down onto PIPAAm coated glass coverslips to allow for ECM coated stamps to adhere. At step 135, HUVECs are seeded onto coverslip after stamp is removed and allowed for adhere for 24 hours at 37° C. At step 145, room temperature media are added to the coverslips to allow for the thermally sensitive PIPAAm to dissolve. At step 155, the ECM rectangles are released and wrap around the cells. Each of these steps is subsequently described in greater detail.

Spin-Coating PDMS and PIPAAm onto Glass Coverslips

Glass coverslips were sonicated in a 50% v/v solution of ethanol to remove any particulates. The coverslips were then dried using nitrogen spray gun and placed in a large Petri dish. The PDMS used to spin-coat these coverslips was prepared by using a 1:10 ratio of Sylgard 184 to curing agent. This solution was then mixed until homogenous and put into a vacuum chamber to remove bubbles for 45 minutes. The coverslips were then added to the spin coater with a 200 μL droplet of PDMS placed in the center of the coverslip. The coverslips were spin-coated for 2 minutes at 2000 rpm. PDMS coated coverslips were cured in 65 degrees Celsius for 4 hours. Once complete, the coverslips were stored until further use.

Culturing HUVECs

HUVECs were taken from the liquid nitrogen room, allowed to thaw in the water bath, combined with 10 mL growth media, spun down to a pellet, and then aspirated and combined with more growth media to reach a concentration of 10⁶ cells/mL. The cells were then allocated at a concentration of 5,000 cells/cm² into 175 cm² flasks. The media was replaced every other day until cells reached confluency approximately 80% confluency (at which point they were used for experiments).

Stamp onto Coated Coverslips Using Fibronectin

Fibronectin was prepared by initially creating a 50 μg/mL solution of unlabeled Fibronectin. This was done by combining 3.8 mL of deionized water with 0.2 mL of 1 mg/mL unlabeled fibronectin. From there, a 10% labeled Fibronectin solution was created through combining 900 μL of the 50 μg/mL solution of unlabeled Fibronectin, 92.5 μL of deionized water, and 7.5 μL of 667 μg/mL labeled 633-Fibronectin.

The PDMS stamps were added to a beaker filled with a solution of 50% ethanol solution and 50% distilled/deionized water. This beaker was placed in the sonicator for at least 30 minutes. The stamps were then picked up individually using sterile tweezers and dried using the nitrogen gun. The stamps were then placed in a sterile Petri dish to await Fibronectin coating. 300 μL of the Fibronectin solution was then pipetted onto each PDMS stamp with the stamp facing upwards, as shown in step 115. Using the pipet, fibronectin was spread to all four corners of the PDMS stamp and allowed to coat the stamps for at least one hour.

Nearing the end of this hour, the PDMS coated coverslips were placed in the UVO cleaner for 15 minutes with the lid placed below the dish inside the cleaner. Immediately after the 15 minutes, the Petri dish was placed within the biohood to maintain sterility. Using tweezers, each of the coverslips were placed into their own well of a 6-well culture plate.

The PDMS stamps were washed by quickly swirling them individually in a dish of deionized water and then washed once more in another dish of deionized water. The stamps were then dried using the nitrogen gun and then using a second pair of forceps to ensure good contact, the stamps were flipped stamp facing down onto the PDMS-coated coverslips, as shown in step 125. The stamps were gently tapped to ensure good contact with the coverslips. The stamps were then gently lifted off the coverslips without twisting to avoid disrupting the transferred pattern. The coverslips were then submerged in 1×PBS for storing purposes.

The protocol for stamping PIPAAm coated coverslips only differs in certain steps from the above protocol. This includes leaving the lid on the Petri dish during the UVO treatment and not using PBS for storage. Generally, stamps are left on the coverslips for at least 45 minutes and up to 24 hours.

Seeding onto PDMS Stamped coverslips As shown in step 135, the stamped coverslips' PBS was aspirated and then seeded with the HUVEC concentrations of 150,000 cells/cm², 300,000 cells/cm², and 450,000 cells/cm².

Seeding onto PIPAAm Stamped coverslips In an aspect, at step 135, a heat-block was sprayed down with 70% ethanol and placed in the biohood. The left side was set to 52 degrees Celsius and the right side to 45 degrees Celsius. Stamped PIPAAm coverslips were secured to the bottom of their own Petri dishes using vacuum grease. The Petri dishes were then placed on the left side of the heat-block. Each coverslip needs 2 mL of fluid. The intended experimental cell concentrations were added in 15 mL tubes and placed in the right side of the heat-block for approximately five minutes or until a temperature between 38 to 40 degrees Celsius is accomplished. Coverslips achieved a temperature between 38 to 40 degrees Celsius. 2 mL of the cell solution was then added to each coverslip and placed immediately in the incubator to prevent any decreases in temperature that would cause the PIPAAm to prematurely dissolve.

Thermally Releasing the Substrates to Form the Endothelial Tubules

As step 145, the PIPAAm was thermally released through the process of adding room temperature media to the dishes while under the microscope. The cells were fixed through aspirating the media, washing with 1×PBS with calcium and magnesium, aspirating once more, ensuring that no cells are being aspirated, and then adding 4% Formaldehyde in the hood and waiting 30 minutes, at which point, the solution is aspirated carefully and the cells are submerged in 1×PBS and then stored.

The coverslips were the primary ZO-1 antibody (5:200 ratio) and then washed in 1×PBS three times with 30-minute increments in between. The coverslips were then stained with DAPI (1:100 ratio), 488 Phalloidin (3:200 ratio), and secondary goat anti-mouse 555 (5:200 ratio). The ZO-1 antibody stains for tight junctions between cells. These tight junctions are an indicator of high cell confluency which is vital in the formation of endothelial tubule segments.

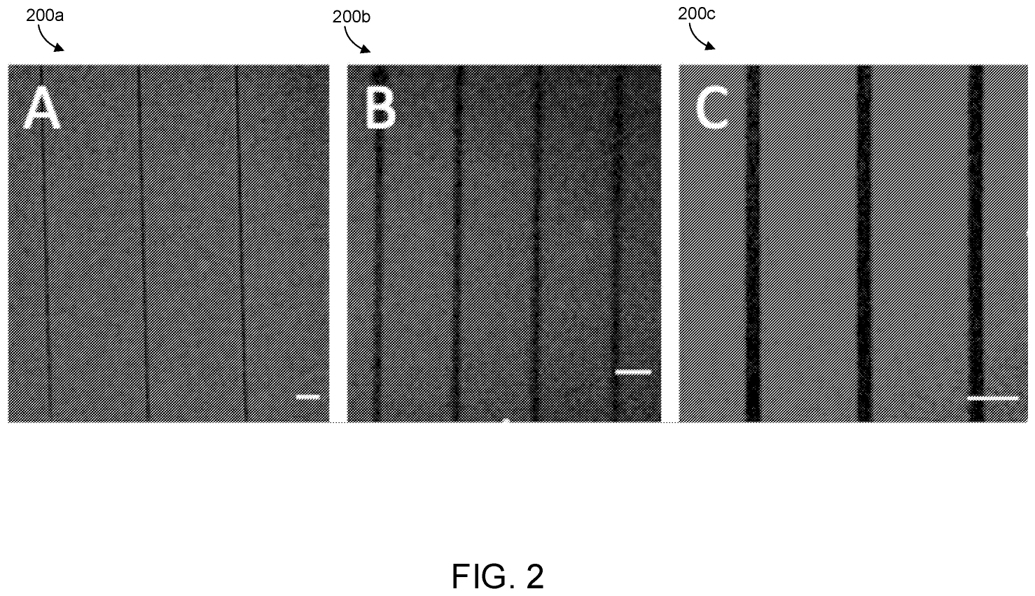

Turning to FIG. 2, images 200a-c show fibronectin stamped on PDMS coated coverslips with the following dimensions. In image 200a, the dimensions include 200×10 μm rectangles. In image 200b, the dimensions include 200×20 μm rectangles. In image 200c, the dimensions include 200×30 μm rectangles. The scale bar is 50 μm for each of these images. These examples show that it is possible to accurately pattern micropattern fibronectin rectangles onto a PIPAAm substrate.

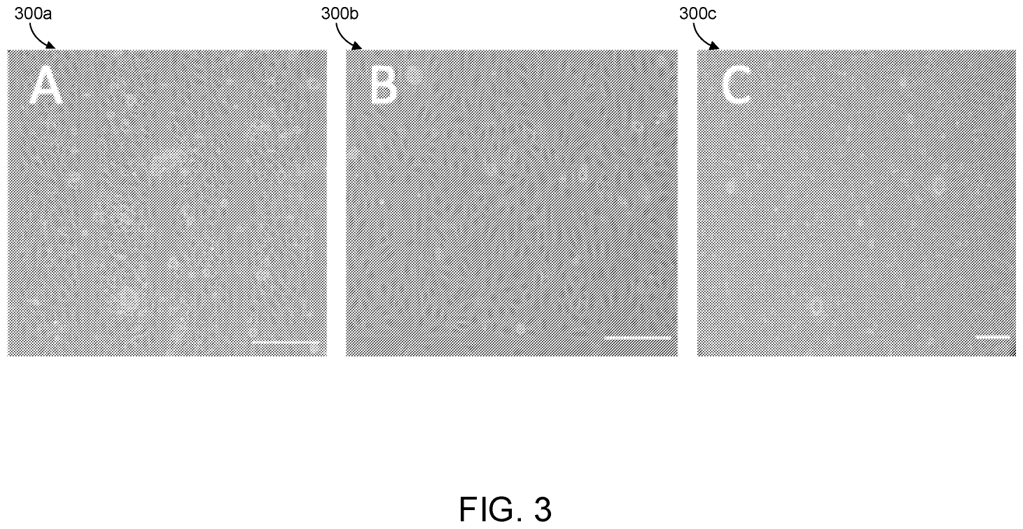

FIG. 3 shows images 300a-c of HUVECs seeded on 200×10 μm stamped PDMS coated coverslips at different cell concentrations. Image 300a shows a concentration of 150,000 cells/cm². Image 300b shows a concentration of 300,000 cells/cm². Image 300c shows a concentration of 450,000 cells/cm². Here, the scale bar is 500 μm for each image. Generally, different seeding concentrations of human umbilical endothelial cells (HUVECs) were used to assess the ability of endothelial cells to attach to the micropatterned fibronectin rectangles and create a confluent monolayer on them within 24 hours. As the seeding concentration of HUVECs increased, there was an increase in endothelial cells bridging between rectangles. This was especially noticeable in the stamps where rectangles were only separated by a small distance of 10 μm (200×10 μm patterns). However due to the higher concentration of cells, the rectangles became indistinguishable, indicating that the 200×10 μm rectangles are too close together for seeding with higher concentrations of HUVECs.

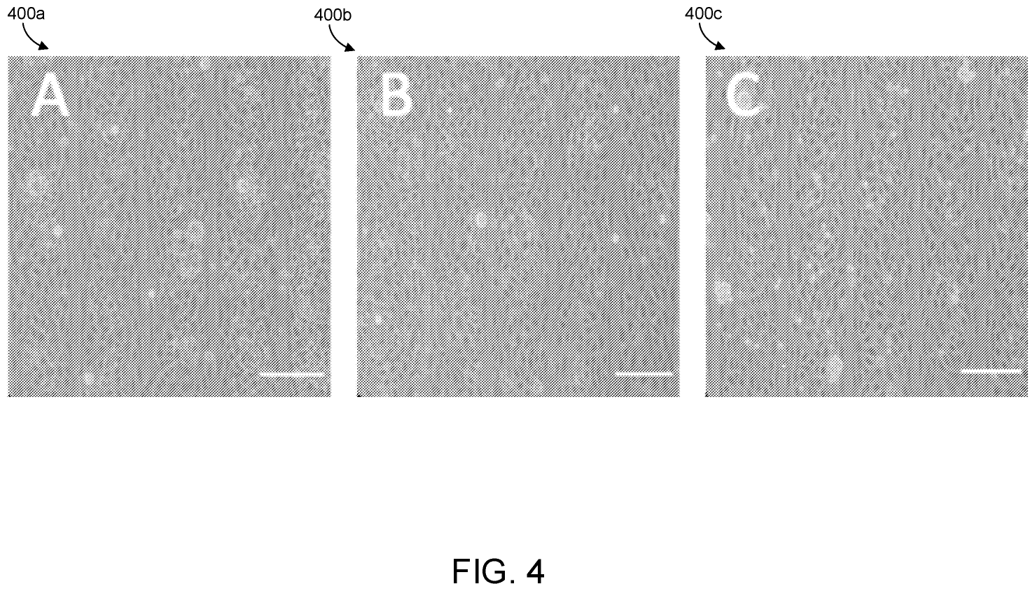

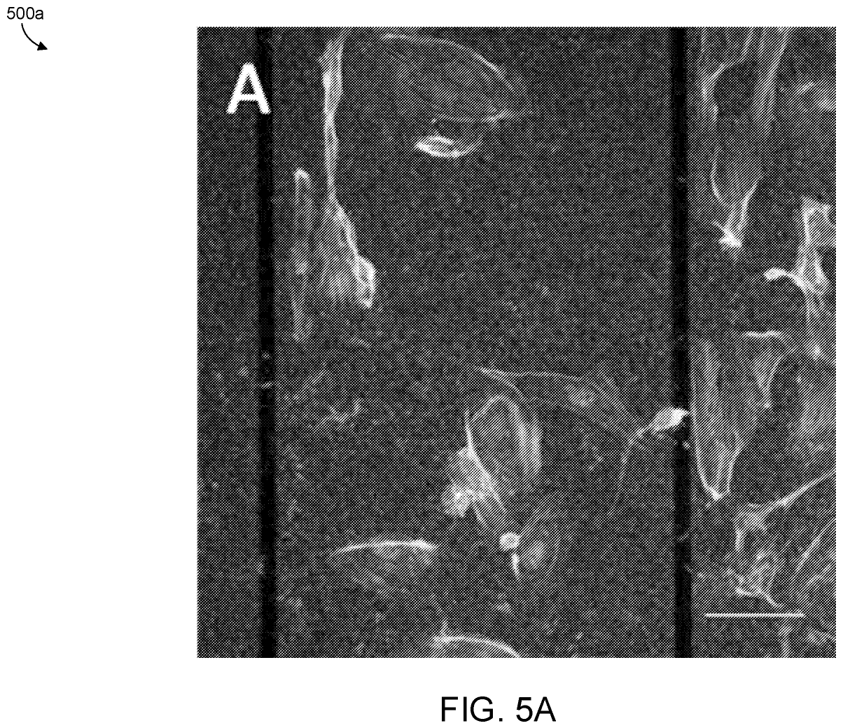

FIG. 4 shows images 400a-c of HUVECs seeded on the 200×20 μm stamped PDMS coated coverslips at different cell concentrations. Image 400a shows a concentration of 150,000 cells/cm². Image 400b shows a concentration of 300,000 cells/cm 2. Image 400c shows a concentration of 450,000 cells/cm². In images 400a-c, the scale bar is 500 μm. In contrast with images 300a-c, with more spacing between adjacent rectangles (200×20 μm), as the cell concentration increased, there is minimal overlap and the rectangles maintain their distinct shape. The cells are very confluent, which is an aspect that helps for enabling the endothelial lining of our microvasculature in vivo.

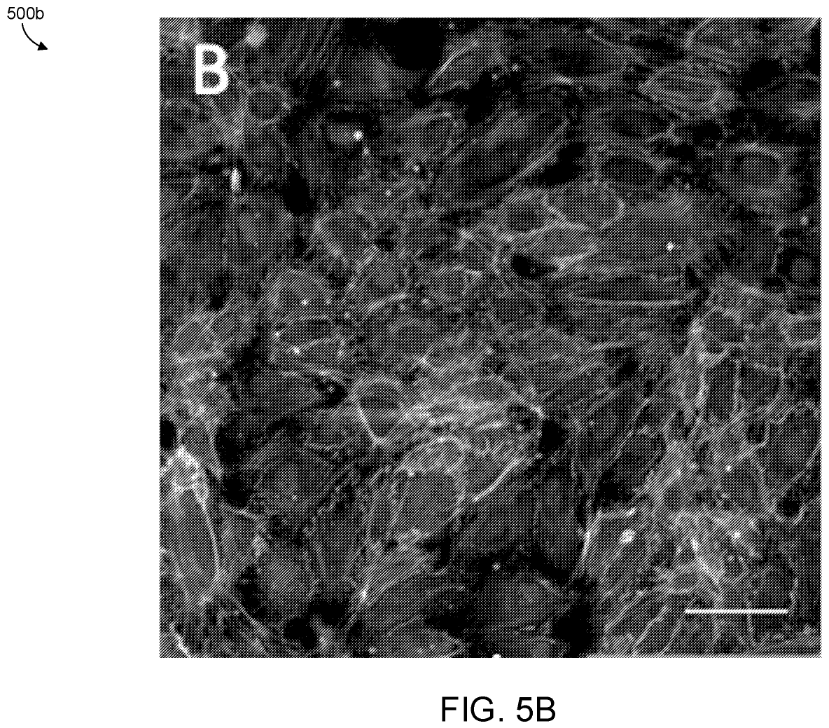

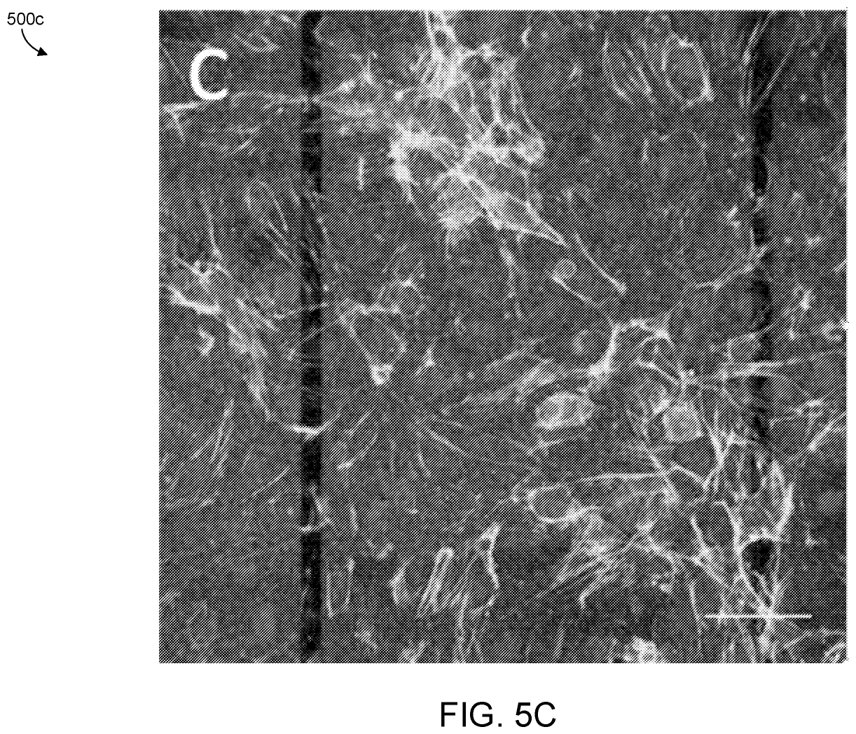

FIGS. 5A-5C each show confocal images 500a-c of HUVECs seeded on the 200×10 μm stamped PDMS coated coverslips at different cell concentrations. Image 500a of FIG. 5A shows a cell concentration of 150,000 cells/cm². Image 500b of FIG. 5B shows a cell concentration of 300,000 cells/cm². Image 5C shows a cell concentration of 450,000 cells/cm². The coverslips were stained with DAPI (blue), Phalloidin (green), and ZO1-antibody (red) for tight junction staining. The stamped fibronectin can be seen in a darker shade (e.g., magenta) in images 500a and 500c. In each image 500a-c, the scale bar is 50 μm.

As previously mentioned, tight junctions are important junctional components present between endothelial cells within the tissue microvasculature. To determine whether tight junctions are present in HUVECs seeded on the micropatterned rectangles, an antibody is used that is targeted against ZO-1. This antibody is a marker of tight junctions. In samples with lowest seeding density, very low levels of ZO-1 was observed, as shown in image 500a of FIG. 5A. This is due to low cell-cell contact in low density seeded samples. Tight junctions between cell borders are readily apparent in high density monolayers in image 500b of FIG. 5B. A “no-primary antibody” control is shown in image 500c of FIG. 5C to demonstrate the specificity of the antibody toward ZO-1 tight junctions. The lack of tight junction staining in image 500c shows that the ZO-1 antibody staining was effective in image 500b.

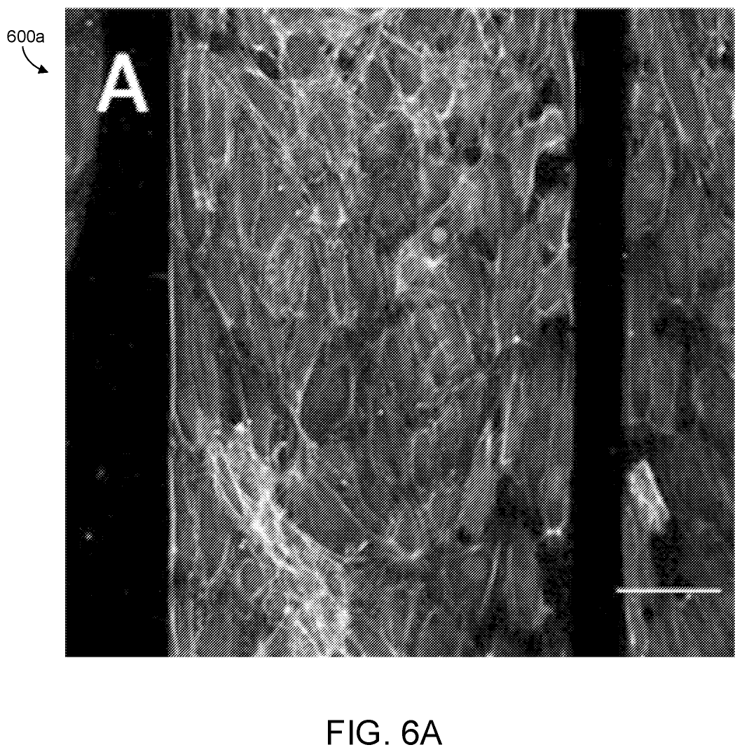

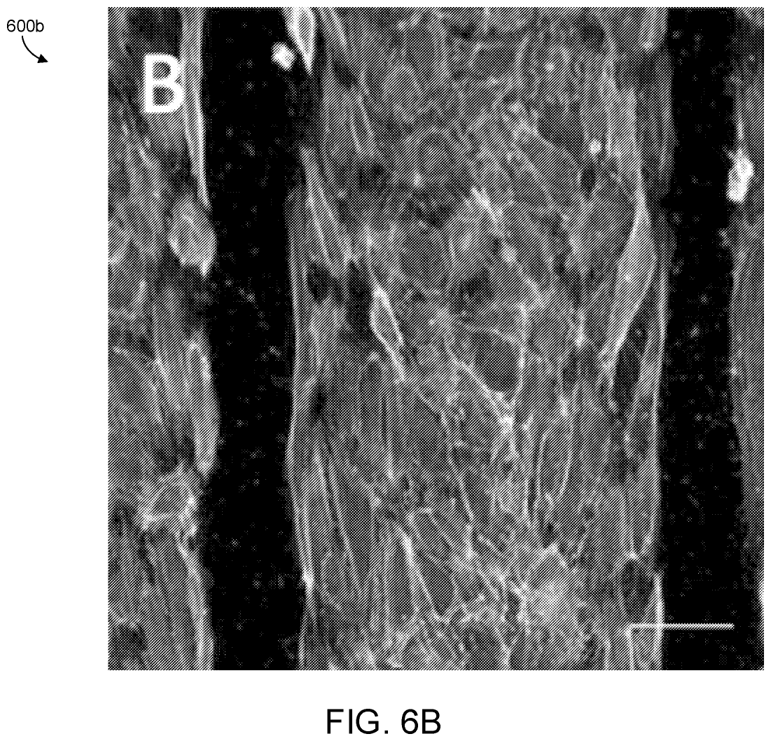

FIGS. 6A-6B show confocal images 600a-b of HUVECs seeded on the 200×20 μm stamped PDMS coated coverslips at different cell concentrations. Image 600a of FIG. 6A shows a cell concentration of 150,000 cells/cm². Image 600b of FIG. 6B shows a cell concentration of 450,000 cells/cm². The coverslips were stained with DAPI (blue), Phalloidin (green), and ZO1-antibody (red) for tight junction staining. In each of images 600a-b, the scale bar is 50 μm. The HUVECs in both concentrations of images 600a-b are very confluent. This can be seen by the large presence of tight junction staining. Despite the increase in cell concentration, the cells are still able to maintain their distinct rectangle patterning.

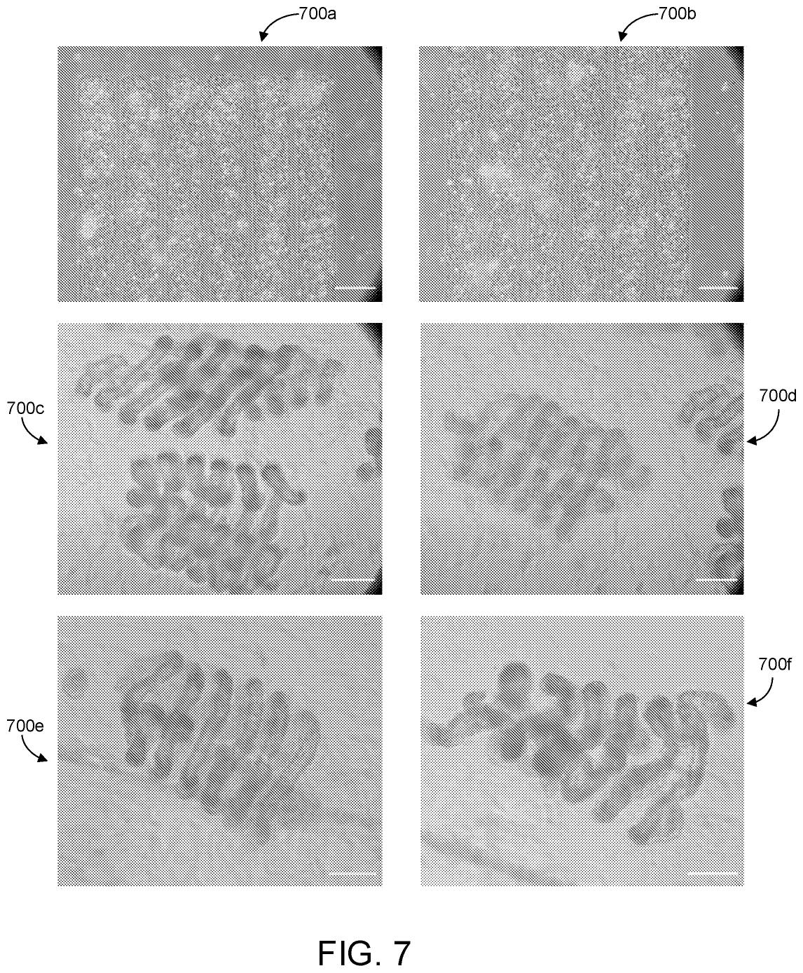

FIG. 7 shows images 700a-f representing time periods of before and after releasing HUVECs seeded on the 200×20 μm stamped PDMS coated with the cell concentration of 300,000 cells/cm 2. To test if HUVECs seeded on micro-patterned rectangles can be released, we patterned 200×20 μm rectangles onto PIPAAm-coated coverslips, and seeded HUVECs onto them. Prior to release HUVECs, seeded on rectangles demonstrated high confluency and distinct attachment to micro-patterned rectangles. Each of images 700a-f represent a same coverslip. In images 700a-f, the scale bar is 500 μm. As seen in image 700a, the corners of the rectangles are all well-defined and show how the effective the seeding was in terms of fully coating the fibronectin stamps. Following PIPAAm release in images 700c-f, the HUVECs were able to maintain their individual patterns and show signs of curling which may suggest they were able to form cylinder-like segments in three-dimensions.

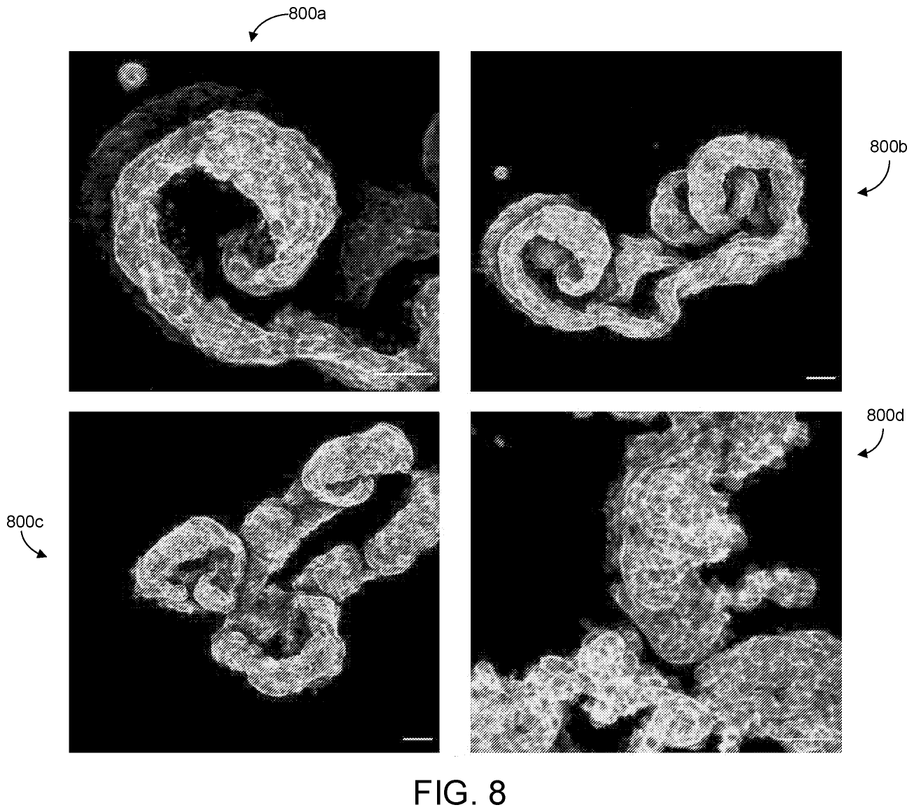

FIG. 8 shows images 800a-d of HUVECs seeded on the 200×20 μm stamped PDMS coated coverslips with the cell concentrations of 300,000 cells/cm² that are released, stained, and imaged. Each of images 800a-d represent a same coverslip. The coverslips were stained with DAPI (dark), Phalloidin (light). Images 800a-c are taken at 10× magnification. Image 800d is taken at 20× magnification. For each image, the scale bar is 50 μm. The confocal imaging of released tubular segments show that the released rectangles maintain their individual segments and form tube-like structures. These structures can be seen to curl into themselves slightly in each of images 800a-d.

In an aspect, based on the images previously described, an example optimal cell concentration is about 300,000 cells/cm². Generally, 450,000 cells/cm² was too high in all three micro-patterned dimensions. The 200×20 μm micro-patterned rectangles have the most confluent cells compared to the 200×10 μm size, which tended to have cells spread out beyond the micro-patterned rectangles.

### Example Embodiment: Wrapped μMonolayers of Corneal Endothelial Cells

The CE is the single layer of cells that lines the posterior surface of the cornea and is responsible for maintaining proper corneal thickness and clarity by pumping excess fluid out of the stroma into the anterior chamber. The cells of the CE are arrested in the GI phase of the cell cycle and therefore cannot replicate to repair damage due to disease, injury, or normal aging. As a result, when cells die, the remaining cells become larger to maintain the integrity and pump function of the monolayer, decreasing the cell density. Once the cell density drops below ˜500 cells/millimeter (mm)², the CE can no longer properly function, causing excess fluid to build up in the corneal stroma and resulting in corneal blindness. The structure (cell mono layer), function (barrier/fluid pumping), and location (adjacent to the anterior chamber) of the CE make it an ideal target for cell injection therapy compared to more complex tissues. Still, animal models and clinical studies have shown that injection in to the anterior chamber for CE repair suffers from the lack of cell retention, viability and integration into the CE, requiring the injection of large numbers of cells and small molecules such as the ROCK inhibitor Y-27632, in order to achieve desirable results. However, even the best results are still limited in their regenerative capabilities, which is because cell-cell junctions and cytoskeletal structure, for CE cells in particular, have been shown to be necessary for cell signaling, function and maintenance of a mature, non-proliferative monolayer state through contact inhibition.

This embodiment describes shrink-wrapping (e.g., encapsulation) of micron-scale monolayers of CE cells (CECs) in the nanometer thick ECM scaffolds according to the encapsulation method described in relation to FIG. 1A. Upon shrink-wrapping, micron-scale monolayers (μMonolayers) contract to size that is small enough to be injected through a 300 gauge needle while maintaining a well-organized monolayer cell sheet with cell-cell junctions and cytoskeletal structure. In vitro, shrink-wrapped μMonolayers significantly increase the cell density of existing monolayers compared to single cells encapsulation.

This description describes experimental results in which shrink-wrapped μMonolayers were injected into the anterior chamber of rabbit eyes in vivo to demonstrate in vivo integration into a CE to increase cell density. Experimentation has shown that shrink-wrapping CEC μMonolayers in ECM scaffolds maintains cell cytoskeletal structure, tight junctions and viability post-injection.

To engineer monolayers of CECs with micron scale dimensions (μMonolayers), modifications were made for shrink-wrapped single cells (termed SHELLs) in nanometer thick ECM scaffolds. Here, the dimensions of the squares that are patterned on poly(N-isopropylacrylamide) (PIPAAm) via surface-initiated assembly techniques are increased in size, to ˜200 μm×200 μm. The increased size of the scaffolds allows for more cells to adhere to each scaffold. The culture time of the cells on the squares to 24 hours is increased to approximately 24 hours. Because PIPAAm is thermoresponsive, samples and cells were heated to 40° C. during the seeding process to ensure the PIPAAm did not dissolve.

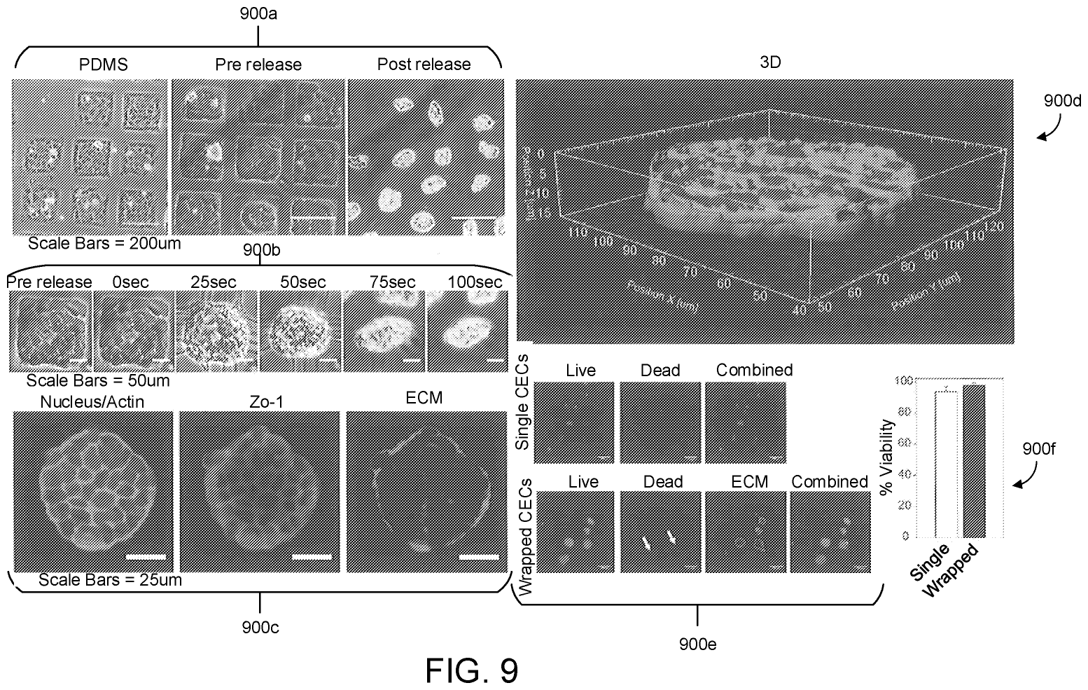

Turning to FIG. 9, examples of cultured cell monolayers are shown. As a control, the same size scaffolds were micro-contact printed onto PDMS, as shown in images 900a. CECs form mono layers on ECM squares microcontact printed onto PDMS (used as a control) and on the thermoresponsive polymer PIPAAm. Once the PIPAAm is dissolved the CEC monolayers contract and are shrink-wrapped in the ECM squares. After 24 hours, Bovine CEC (BCECs) μMonolayers cultured on ECM scaffolds fabricated on PIPAAm exhibited similar morphology to those cultured on ECM scaffolds fabricated on PDMS, when viewed under phase microscopy.

Images 900b show that the release and shrink-wrapping of the CECs occurs quickly, in <100 seconds once the sample cools to room temperature. Upon dissolution of the PIPAAm and thermal release, the μMonolayers remained intact and were successfully shrink-wrapped within the ECM squares. Once released, the shrink-wrapped μMonolayers were collected, centrifuged, injected through a 280 needle and allowed to settle for 30 min before fixing and staining to investigate the structure of the BCECs within the μMonolayers. As seen in images 900c, shrink-wrapped BCECs exhibited continuous ZO-1 at the borders and a cortical F-actin structure indicating that the cells maintained their tight-junctions and cytoskeletal structure throughout the release process. Confocal microscopy images show that after injection, the CECs maintain both their cytoskeletal structure (F-actin) and tight junctions (ZO-1).

Image 900d includes a 3D projection of a shrink-wrapped CEC monolayer 30 minutes after injection illustrating how it begins to relax and return to its original shape. Image 900d shows that post-release, the shrink-wrapped μMonolayers began to relax and return to their as engineered square like structure approximately 30 minutes after injection. Viability of the cells within the μMonolayers after injection was examined using a live/dead cytotoxicity assay and compared to enzymatically-released single cells. Image 900e shows representative live/dead images of control single CECs and shrink-wrapped CECs show that both types of cells are viable with very few dead cells present. Confocal microscopy images indicated that the only dead cells in the shrink-w rapped μMonolayer samples were individual cells that were not integrated into μMonolayers.

Live/dead data (n=3) was statistically compared using a Student's t-test and no significant difference in viability between single cells and shrink-wrapped cells injected through a 28G needle was observed with both types exhibiting over 90% viability. Image 900f shows that quantification determined that single CECs had greater than 93% viability and shrink-wrapped cells (including the cells not integrated within the μMonolayers) had greater than 97% viability.

Shrink-wrapped μMonolayers display different growth characteristics compared to single CECs. To assess the potential use of shrink-wrapped μMonolayers in cell injection therapy, it was determined whether they would attach to and proliferate on a denuded stroma, which is primarily collagen I. Both single CECs and shrink-w rapped μMonolayers were injected onto compressed collagen I gels that served as a denuded stromal mimic. Samples were fixed and stained at 6 and 24 hours post-inject ion to observe the cell structure and outgrowth.

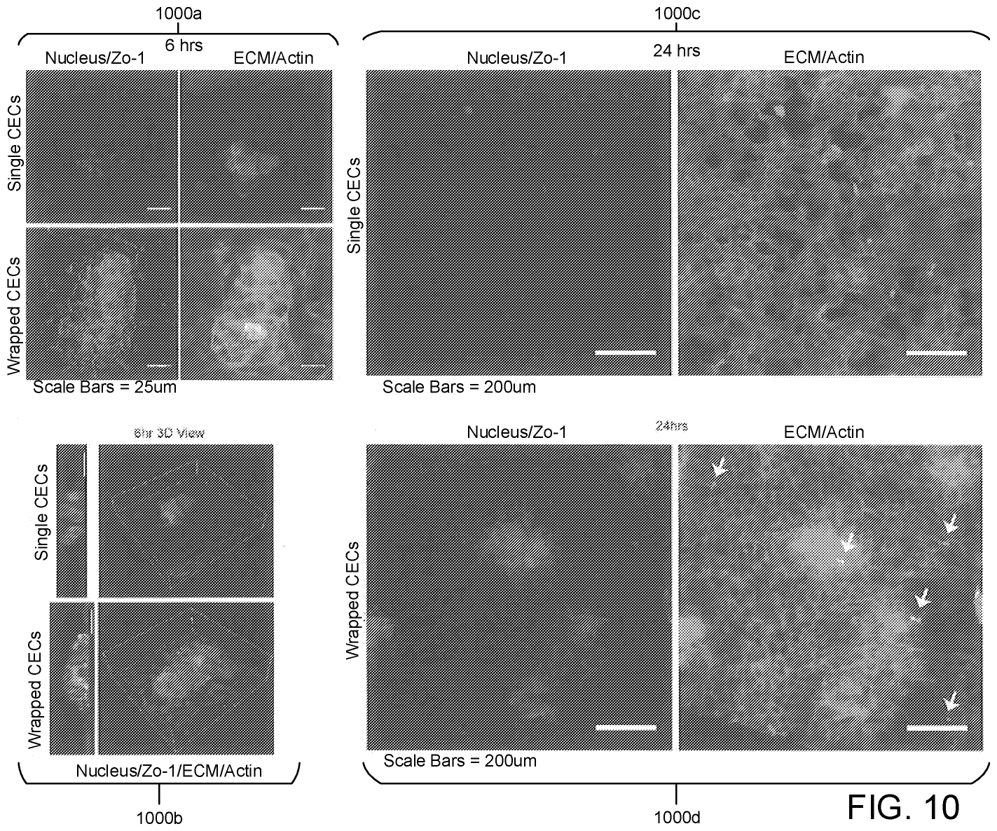

Turning to FIG. 10, as shown in images 1000a, six hours post injection, single CECs were mostly circular with very little spreading observed. The F-actin staining showed a lack of filamentous structure to the cytoskeleton of the cells. Additionally, there was no ZO-1 observed. In contrast, the CECs from the shrink-wrapped μMonolayers maintained their cytoskeletal structure and tight-junctions, as evidenced by the F-actin filaments and continuous ZO-1 expression at the cell borders (two bottom images of 1000a). More specifically, six hours after reseeding onto a collagen I gel, CECs maintained ZO-1 expression and F-actin cytoskeleton, while growing out of the ECM scaffolds. The cells at the periphery of the shrink-wrapped CECs are also expressing ZO-1. In contrast, the single CECs have no established F-actin cytoskeleton or ZO-1 expression.

Images 1000b show a comparison between single CECs (top image) and wrapped monolayers (bottom image). The 3D views of the cells at 6 hours post seeding, show the differences between the single CECs and shrink-wrapped CECs. Examining the samples in 3D confirmed that single CECs were very rounded and had very little interactions between cells, whereas the shrink-wrapped uMonolayers had inverted with the ECM scaffolds now present within the center of the cells which were directly attached to the stromal mimic (1000b, bottom). After 24 hours, the single CECs covered most of the stromal mimic and had a more defined cytoskeletal structure compared to the CECs at 6 hours with many F-actin stress fibers across the cell bodies. Images 1000c show that, at 24 hours, single CECs have begun to spread and cover almost the entire scaffold. At 24 hours, the CECs have already grown out of the ECM scaffolds and formed an almost complete monolayer. In images 1000d, the nucleus is dark (shaded blue), ZO-1 is light (shaded red), COL4 is shaded magenta, and F-actin is shaded green. The scale bars are 50 μm except in the orthogonal views, in which the scale bars are 20 μm. The single CECs exhibited very little, discontinuous ZO-1 at the cell borders. In contrast, the shrink-wrapped uMonolayers had continuous ZO-1 at all cell borders and cortical F-actin cytoskeleton, which closely resembled the structure of in vivo CECs, as shown in images 1000d. The ECM scaffolds were still visible after 24 hours as indicated by the arrows in image 1000d (right image).

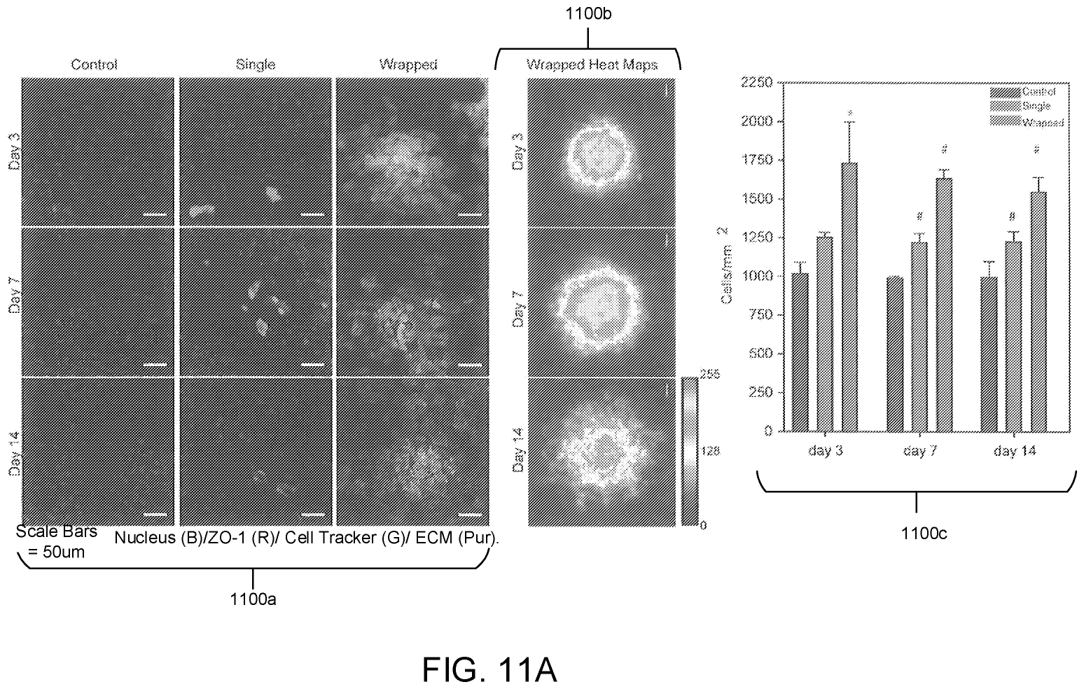

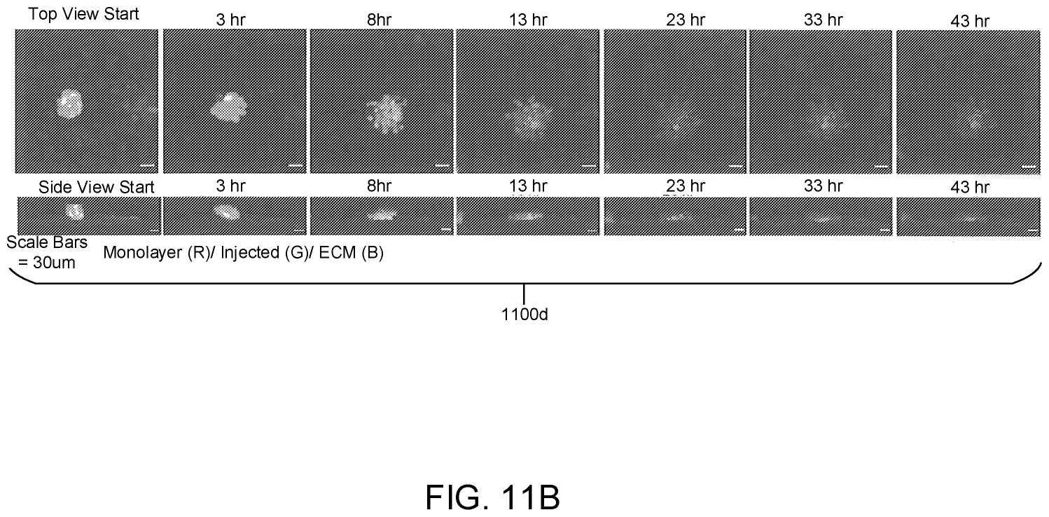

Shrink-wrapped uMonolayers integrate into CE monolayers and increase density in vivo. Many patients need corneal transplants due to failure caused by low cell density and not disease and therefore could benefit from an injection of cells to boost cell density without a complete replacement. Therefore, the integration of shrink-wrapped CECs into existing CE monolayers is performed. Low density CE monolayers were formed by seeding late passage BCECs on the collagen I gel stromal mimics used in the previous experiments. Shrink-wrapped uMonolayers or the equivalent number of single CECs were then injected through a 300 needle on to confluent CE monolayers. The CECs used for both the single cell and uMonolayers injections were labeled with CellTracker green to track the cells post-injection and CE monolayers that were not injected with any cells served as controls. Samples were fixed, stained and analyzed at 3, 7 and 14 days post injection. Images of each of these data points are shown in FIG. 11A in grid 1100a. At each time point, very few single CECs could be seen integrated into existing monolayers. At day 3, the shrink-wrapped μMonolayers that integrated appeared to be very densely packed compared to the cells from the CE monolayer and still had some 3D structure to them. By day 7, the shrink-wrapped μMonolayers had completely integrated into the mono layer, but the CellTracker positive cells from the μMonolayers appeared smaller than those from the existing monolayer. Images 400a are labeled to show control cells, single cells, and wrapped cells. At day 14, the shrink-wrapped μMonolayers now appeared to be of similar size to the cells from the existing monolayer indicating that the integration and density equilibration of the monolayer had been achieved. Cell Tracker labeled single cells and shrink-wrapped cells were visible at each time point. However, significantly more shrink-wrapped cells were present at each time point, and the ECM scaffolds were still visible 14 days after injection.

Confocal images of shrink-wrapped μMonolayers integrating at each time point were quantified using a heat map representation. The average pixel density of the CellTracker signal for each integrating μMonolayer was calculated and results are displayed in the heat maps shown in images 1100b. These heat maps confirm the observation that the shrink-wrapped CEC μMonolayers initially integrate in a densely packed cluster that spreads out over time. The heat maps of the area occupied by Cell Tracker labeled shrink-wrapped cells show that over the course of 14 days indicate that the cells initially integrate in a tight cluster and then the density starts to equilibrate as the cells spread out slightly. For example, for day 3 the number is 33, for day 7, the number is 37, and for day 14, the number is 40.

To quantify if shrink-wrapped μMonolayers increased the cell density of the CE monolayers, confocal images were obtained and the cell density was determined by counting the nuclei per image and dividing by the image area for each sample type at each time point. Ten images per sample were counted to obtain an average per sample and the average per sample type was calculated from either 3 or 4 individual samples and results are shown in graph 1100c. At day 3, the CE monolayers with integrated shrink-wrapped μMonolayers (1731±267 cells/mm 2 had a significantly higher cell density compared to controls (1016±75 cells/mm 2) but there was no significant difference compared to CE monolayers with integrated single CECs (1253±31 cells/mm 2). At day 7, the CE monolayers shrink-wrapped μMonolayers integrated (1631±58 cells/mm 2) had a significantly higher density compared to both controls (989±11 cells/mm 2 and monolayers with single CECs integrated (1220±56 cells/mm 2). Similarly, at day 14, the CE monolayers with shrink-wrapped μMonolayers integrated (1545±95 cells/mm2 had a significantly higher density compared to both controls (994±104 cells/mm 2) and monolayers with single CECs integrated (1224±66 cells/mm). The large standard deviation in cell density at day 3 observed in the shrink-wrapped samples decreased at day 7 and 14 further confirmed the observations made qualitatively from the confocal images and heat maps. This continued integration and equilibration also explains the perceived decrease in cell density from day 3 to 14, where the cell density overall isn't necessarily decreasing, it is just becoming more homogenous across the CE monolayer.

Although these long-term results are useful, it was also important to understand the short-term integration of the shrink-wrapped CECs to determine if method used clinically for single cell injections, keeping the patient face down for 3 hours, would be sufficient for injection of shrink-wrapped μMonolayers. Image 1100d of FIG. 11B shows time lapse images from live confocal imaging of the integration of shrink-wrapped CECs into an existing mono layer. At 3 hours, the cells have attached and begun to integrate and by 43 hours the cells are almost completely integrated into the mono layer. Live confocal imaging of the integration of a shrink-wrapped μMonolayer was performed by labeling the shrink-wrapped cells with Cell Tracker Green and the cells in the CE monolayer with Cell Tracker Orange. A Z-stack was collected every hour for 48 hours and the time lapse video is seen in Supplemental video 51. At 3 hours post injection, the shrink-wrapped μMonolayer had begun to attach and flatten on top of the CE mono layer. The CE monolayer cells continued to move over the course of the 48 hours to allow for the shrink-wrapped μMono layer to integrate. By 43 hours, the shrink-wrapped μMonolayer was almost completely 20 and integrated into the CE monolayer. This video also confirmed the previous results that the cells within the shrink-wrapped μMonolayers are very densely packed compared to the CE monolayer cells at early time points. Shrink-wrapped μMonolayers begin to integrate into ex vivo rabbit corneas within 3 hours. Although the in vitro live imaging results suggest that having a patient lie face down for 3 hours would be a sufficient amount of time post-injection for the shrink-wrapped μMonolayers to integrate, the in vitro CE monolayers potentially have more ability to move to accommodate injected cells compared to an in vivo CE.

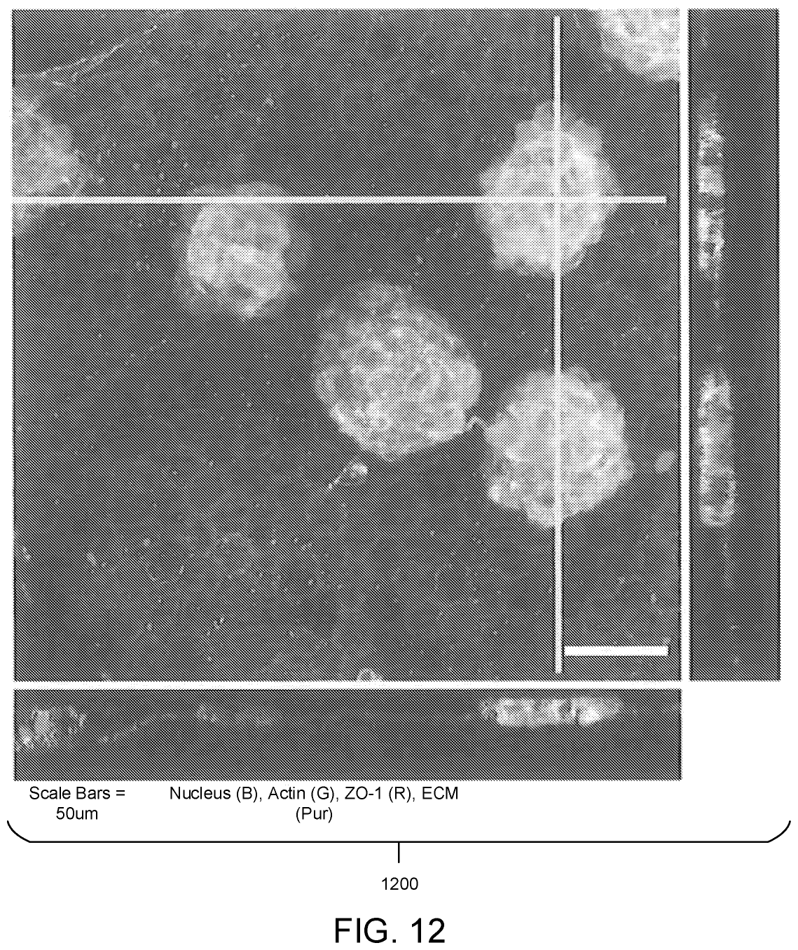

As a next step to confirm these results before moving to in vivo studies, shrink-wrapped rabbit μMonolayers were injected into the anterior chamber of ex vivo rabbit eyes and incubated the eyes cornea down for 3 hours before fixation of the whole globe. Post fixation, the cornea was excised and rinsed vigorously 3 times to remove any non-adhered cells and stained for ZO-1, F-actin and the nucleus. Confocal imaging of the corneas showed numerous shrink-wrapped μMono layers attached over the entirety of the corneas. The shrink-wrapped μMonolayers were still rounded and clustered with the ECM scaffold in the center, as shown in image 1200 of FIG. 12. Confocal images show that the shrink-wrapped cells had begun to integrate into the ex vivo CE and the ECM scaffold is observed to be between the shrink-wrapped cells and the existing monolayers. The vertical and horizontal line indicate the places at which the orthogonal views were obtained.

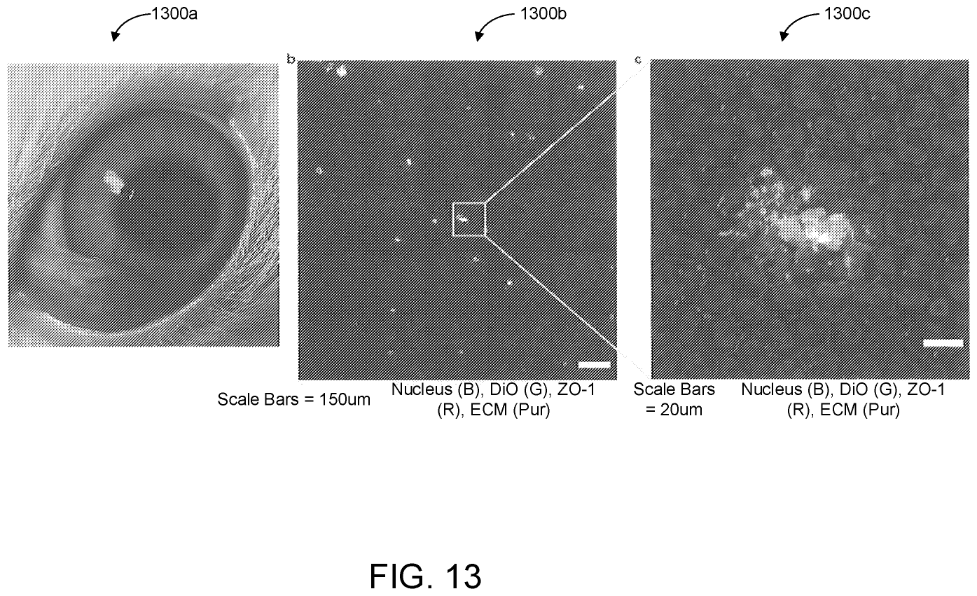

FIG. 13 shows results of shrink-wrapped μMonolayers integrated into healthy rabbit CE in vivo. To test the feasibility of using the shrink-wrapped μMonolayers to increase CE cell density in patients, a study using healthy eyes of 3 New Zealand white rabbits was performed. Shrink-wrapped μMonolayers labeled with DiO were injected into the anterior chamber of one eye of each rabbit. The rabbits were laid on their sides with the injected eye facing down for 3 hours to allow for integration of the shrink-wrapped μMonolayers. The rabbits were then observed for 7 days before sacrifice and enucleation. On day 7, the injected eyes on all 3 rabbits remained clear with no sign visible outward signs of irritation or swelling. Image 1300a is a photograph of the rabbit eye on day 7 shows that the cornea remained healthy and clear. After enucleation, the eyes were fixed via injection of 2% paraformaldehyde (PBS++) into the anterior chamber and immersion of the whole globe in 2% paraformaldehyde (PBS++) for 24 hours before staining for the tight junctions via ZO-1 and the nucleus. Confocal microscopy imaging showed that DiO labeled cells were present in the injected eyes of all 3 rabbits and large tile scans showed numerous clusters of shrink-wrapped μMonolayers integrated throughout the corneas. Image 1300b shows a large area tile scanned confocal image shows many areas with DiO (green) labeled shrink-wrapped cells still present in the cornea 7 days post injection. Higher magnification imaging showed that the cells of the shrink-wrapped μMonolayers had integrated seamlessly with the healthy rabbit CE with ZO-1 present continuously at all cell borders between the DiO labeled cells and the native rabbit CECs. Image 1300c shows a zoomed-in image of the area of 1300b that is highlighted by the box and that shows that the DiO labeled shrink-wrapped cells are integrated into the healthy rabbit CE, exhibiting continuous tight junctions (ZO-1) between the shrink-wrapped CECs and the native CECs. The ECM scaffold is also still visible, as indicated by the arrow and the shrink-wrapped CECs are in the same plane as the monolayer with no cells above the CE indicating seamless integration. The ECM scaffolds were still visible, providing further evidence that the shrink-wrapped μMonolayers had integrated and become a part of the rabbit CE in vivo.

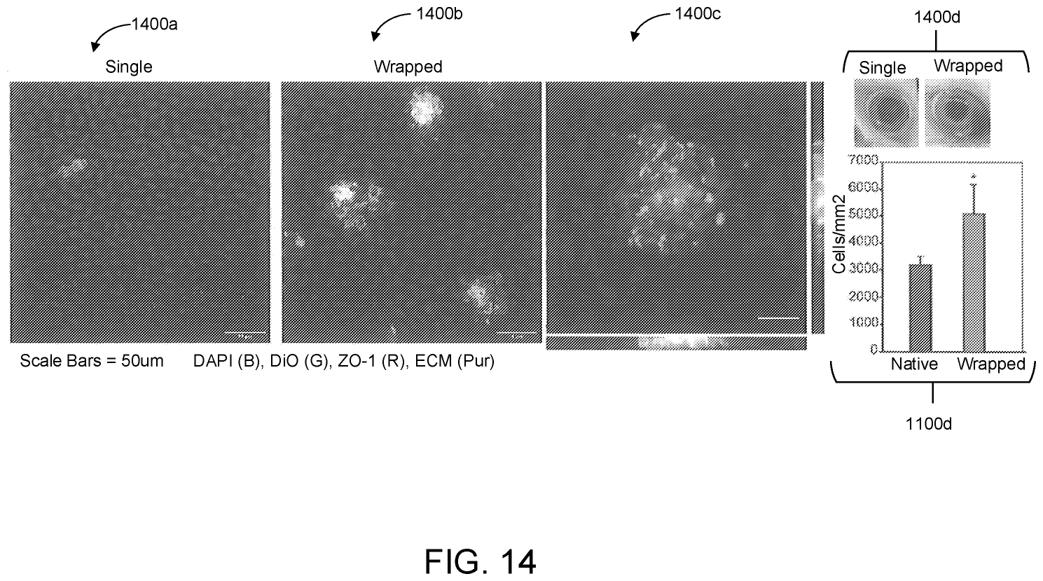

FIG. 14 shows images 1400a-c and graph 1400d that illustrate that shrink-wrapped CECs integrate into and increase local density in a healthy rabbit corneal endothelium. IN image 1400a, very few integrated single cells are in a healthy rabbit endothelium. In contrast, in image 1400b, there are many shrink-wrapped μMonolayers integrated into the healthy rabbit endothelium. Image 1400c shows a close up image of an integrated μMonolayer. The ECM is still central but underneath the cell bodies of the injected cells from the μMonolayers that have completely integrated. Graph 1400d shows an increase in local cell density 7 days post-injection of the area where the μMonolayers have integrated vs other areas of the cornea within the same field of view.

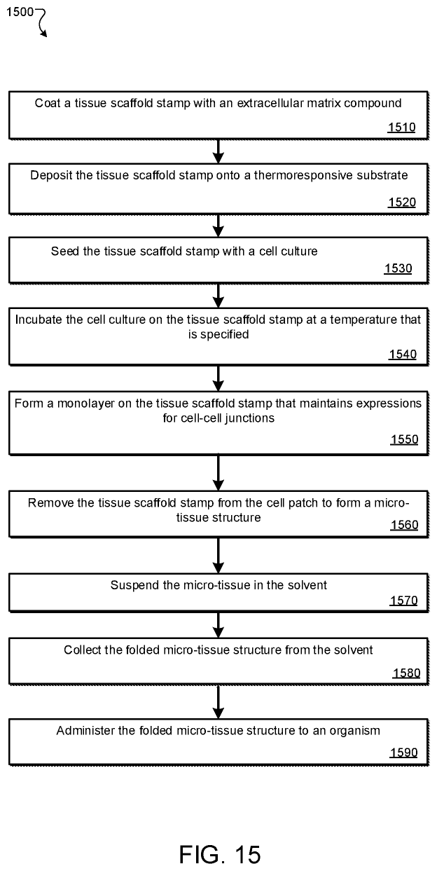

FIG. 15, a process 1500 for encapsulating cells is shown. The tissue scaffold stamp is coated (1510) with an extracellular matrix compound. The tissue scaffold stamp was deposited (1520) onto a thermoresponsive substrate. The tissue scaffold stamp was seeded (1530) with a cell culture. The cell culture was incubated (1540) on the tissue scaffold stamp at a specified temperature. The cell culture forms (1550) a monolayer on the tissue scaffold stamp in which borders of the monolayer maintain expressions for cell-cell junctions, wherein the cell-cell junctions of the monolayer are configured to express tension forces. The thermoresponsive substrate was dissolved by lowering the temperature. The tissue scaffold stamp was removed (1560) from the cell patch to form a micro-tissue structure by dissolving the tissue scaffold stamp in a solvent. The micro-tissue structure was folded (1570) suspending the micro-tissue in the solvent to enable the cell patch to fold the extracellular matrix compound, and the folding of the matrix compound also caused the micro-tissue structure to fold. The folded micro-tissue structure was collected (1580) from the solvent using a centrifuge. The folded micro-tissue structure was then administered (1590) to an organism.

A number of exemplary embodiments have been described. Nevertheless, it will be understood by one of ordinary skill in the art that various modifications may be made without departing from the spirit and scope of the techniques described herein.

What is claimed is:

1. A method for micro-tissue encapsulation of cells comprising:
coating a tissue scaffold stamp with an extracellular matrix compound;
depositing the tissue scaffold stamp onto a thermoresponsive substrate;
seeding the tissue scaffold stamp with a cell culture;
incubating the cell culture on the tissue scaffold stamp at a temperature that is specified, wherein the cell culture forms a cell patch that is attached to the extracellular matrix compound;
forming, by the cell patch, a monolayer on the tissue scaffold stamp in which borders of the monolayer maintain expressions for cell-cell junctions;
removing the thermoresponsive substrate;
removing the tissue scaffold stamp from the cell patch to form a micro-tissue structure around the cell patch;
folding the micro-tissue structure by suspending the micro-tissue in the solvent;
collecting the folded micro-tissue structure from the solvent; and
administering the folded micro-tissue structure to an organism.

2. The method of claim 1, further comprising:
forming the tissue scaffold into a tube configuration; and
forming a cell patch comprising a tube geometry based on the tube configuration of the tissue scaffold.

3. The method of claim 2, wherein the cell patch comprises a fragment of a blood vessel.

4. The method of claim 1, further comprising adding antibodies to the cell patch.

5. The method of claim 1, wherein administering the micro-tissue structure to an organism comprises injecting the micro-tissue structure.

6. The method of claim 5, wherein the cell patch comprises corneal endothelial cells, the method further comprising:
introducing the cell patch to a cornea; and
ensuring a contact of the cell patch with the cornea using gravity.

7. The method of claim 5, wherein a size of the micro-tissue structure is proportional to a size of the tissue scaffold stamp, and wherein the size of the micro-tissue structure is a fraction of a diameter of an injecting apparatus.

8. The method of claim 1, wherein the tissue scaffold stamp comprises an organosilicon compound.

9. The method of claim 8, wherein the organosilicon compound comprises Polydimethylsiloxane.

10. The method of claim 1, wherein the extracellular matrix compound comprises a protein comprising one or more of collagen IV, laminin, a fibroblast growth factor protein, and a vascular endothelial growth factor protein.

11. The method of claim 1, wherein depositing the tissue scaffold stamp comprises printing the tissue scaffold stamp onto the thermoresponsive substrate.

12. The method of claim 1, wherein the thermoresponsive substrate comprises a PIPAAm polymer.

13. The method of claim 1, wherein the tissue scaffold stamp forms a regular geometry.

14. The method of claim 1, wherein the tissue scaffold stamp comprises a surface dimension of less than or approximately equal to 250 μm².

15. The method of claim 1, wherein the cell patch comprises between 10 and 100 cells.

16. A system comprising:
a cell patch comprising a cell monolayer that maintains expressions for cell-cell junctions and cytoskeletons for cells that are in the cell patch; and
a micro-tissue structure folded around the cell patch, the micro-tissue structure comprising an extracellular matrix configured to provide a physical barrier between the cell patch and an external environment.

17. The system of claim 16, wherein the extracellular matrix comprises a protein comprising one or more of collagen IV, laminin, a fibroblast growth factor protein, and a vascular endothelial growth factor protein.

18. The system of claim 16, wherein the micro-tissue structure forms a tube configuration.

19. The system of claim 16, wherein the monolayer comprises between 10 and 100 cells.

20. The system of claim 16, wherein the cell patch comprises muscle tissue, and wherein a growth factor is added to the extracellular matrix to promote vascular ingrowth of the muscle tissue.
